(19) United States
(12) Patent Application Publication
(10) Pub. No.: US 2004/0248919 A1
(43) Pub. Date: Dec. 9, 2004

(54) Fluorinated trienes and their use as rxr modulators

(76) Inventors: Michael Gregory Bell, Indianapolis, IN (US); Timothy Alan Grese, Indianapolis, IN (US); Douglas Linn Gernett, Indianapolis, IN (US); Nathan P. Yumibe, Indianapolis, IN (US); Marcus F. Boehm, San Diego, CA (US); Pierre-Yves Michellys, San Marcos, CA (US); Robert J. Ardecky, Encinitas, CA (US); Christopher M Mapes, San Diego, CA (US); Jyun-Hung Chen, San Diego, CA (US); John S. Tyhonas, Chula Vista, CA (US); Lawrence G. Hamann, Cherry Hill, NJ (US)

Correspondence Address: Steven G Davis, Hamilton Brook Smith & Reynolds, 530 Virginia Road, PO Box 9133, Concord, MA, 01742-9133, US

(21) Appl. No.: 10/471,383

(22) PCT Filed: Mar. 12, 2002
(86) PCT No.: PCT/US02/07718

Related U.S. Application Data
(60) Provisional application No. 60/275,808, filed on Mar. 14, 2001.

(51) Int. Cl. C07C 69/76; A61K 31/40; A61K 31/19; C07D 207/46

(52) U.S. Cl. 514/277; 514/408; 514/568; 546/341; 548/577; 560/55; 562/465

(57) ABSTRACT

The present invention relates to a method of modulating retinoid X receptor activity in a mammal, novel compounds and pharmaceutical compositions for modulating retinoid X receptor activity in a mammal, and methods of making compounds that modulate retinoid X receptor activity in a mammal. The compounds are represented by Structural Formula 1: The compounds of Structural Formual 1 are efficacious insulin sensitizers and do not have the undesirable side effects of increasing triglycerides or suppressing the thyroid hormone axis.

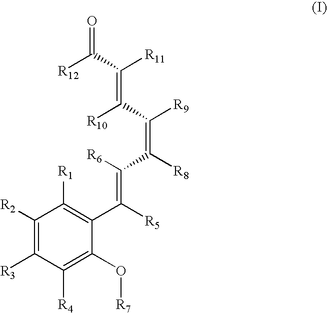

### BACKGROUND OF THE INVENTION

The vitamin A metabolite, retinoic acid, has long been recognized to induce a broad spectrum of biological effects. For example, retinoic acid-containing products, such as Retin-A® and Accutane®, have found utility as therapeutic agents for the treatment of various pathological conditions. In addition, a variety of structural analogues of retinoic acid have been synthesized that also have been found to be bioactive. Many of these synthetic retinoids have been found to mimic many of the pharmacological actions of retinoic acid, and thus have therapeutic potential for the treatment of numerous disease states.

Medical professionals have become very interested in the therapeutic applications of retinoids. Among their uses approved by the FDA is the treatment of severe forms of acne and psoriasis as well as cancers such as Kaposi's Sarcoma. A large body of evidence also exists that these compounds can be used to arrest and, to an extent, reverse the effects of skin damage arising from prolonged exposure to the sun. Other evidence exists that these compounds have clear effects on cellular proliferation, differentiation and programmed cell death (apoptosis), and thus may be useful in the treatment and prevention of a variety of cancerous and pre-cancerous conditions, such as acute promyleocytic leukemia (APL), epithelial cancers, squamous cell carcinomas, including cervical and skin cancers and renal cell carcinoma. Furthermore, retinoids may have beneficial activity in treating and preventing diseases of the eye, cardiovascular disease and other skin disorders.

Major insight into the molecular mechanism of retinoic acid signal transduction was gained in 1988, when a member of the steroid/thyroid hormone intracellular receptor superfamily was shown to transduce a retinoic acid signal. V. Giguere et al., Nature, 330:624-29 (1987); M. Petkovich et al., Nature, 330: 444-50 (1987); for a review, see R. M. Evans, Science, 240:889-95 (1988). It is now known that retinoids regulate the activity of two distinct intracellular receptor subfamilies: the Retinoic Acid Receptors (RARS) and the Retinoid X Receptors (RXRs), including their subtypes, RARα, β, γ and RXRα, β, γ. All-trans-retinoic acid (ATRA) is an endogenous low-molecular-weight ligand that modulates the transcriptional activity of the RARs, while 9-cis retinoic acid (9-cis) is the endogenous ligand for the RXRs. R. A. Heyman et al., Cell, 68:397406 (1992); and A. A. Levin et al., Nature, 355:359-61 (1992).

Although both the RARs and RXRs respond to ATRA in vivo, due to the in vivo conversion of some of the ATRA to 9-cis, the receptors differ in several important aspects. First, the RARs and RXRs are significantly divergent in primary structure (e.g., the ligand binding domains of RARα and RXRα have only approximately 30% amino acid homology). These structural differences are reflected in the different relative degrees of responsiveness of RARs and RXRs to various vitamin A metabolites and synthetic retinoids. In addition, distinctly different patterns of tissue distribution are seen for RARs and RXRs. For example, RXRα mRNA is expressed at high levels in the visceral tissues, e.g. liver, kidney, lung, muscle and intestine, while RARα mRNA is not. Finally, the RARs and RXRs have different target gene specificity. In this regard, RARs and RXRs regulate transcription by binding to response elements in target genes that generally consist of two direct repeat half-sites of the consensus sequence AGGTCA. RAR:RXR heterodimers activate transcription ligand by binding to direct repeats spaced by five base pairs (a DR5) or by two base pairs (a DR2). However, RXR:RXR homodimers bind to a direct repeat with a spacing of one nucleotide (a DR1). D. J. Mangelsdorf et al., “The Retinoid Receptors” in The Retinoids: Biology, Chemistry and Medicine, M. B. Sporn, A. B. Roberts and D. S. Goodman, Eds., Raven Press, New York, N.Y., 2nd Edition (1994). For example, response elements have been identified in the cellular retinal binding protein type II (CRBPII), which consists of a DR1, and in Apolipoprotein AI genes that confer responsiveness to RXR, but not to RAR. Further, RAR has also been shown to repress RXR-mediated activation through the CRBPII RXR response element (D. J. Manglesdorf et al., Cell, 66:555-61 (1991)). Also, RAR specific target genes have been identified, including target genes specific for RARβ (e.g., βRE), that consist of a DR5. These data indicate that two retinoic acid responsive pathways are not simply redundant, but instead manifest a complex interplay. RXR agonists in the context of an RXR:RXR homodimer display unique transcriptional activity in contrast to the activity of the same compounds through an RXR heterodimer. Activation of a RXR homodimer is a ligand dependent event, i.e., the RXR agonist must be present to bring about the activation of the RXR homodimer. In contrast, RXR working through a heterodimer (e.g., RXR:RAR, RXR:VDR) is often the silent partner, i.e., no RXR agonist will activate the RXR-containing heterodimer without the corresponding ligand for the heterodimeric partner. However, for other heterodimers, (e.g., PPAR:RXR) a ligand for either or both of the heterodimer partners can activate the heterodimeric complex. Furthermore, in some instances, the presence of both an RXR agonist and the agonist for the other heterodimeric partner (e.g., gemfibrizol for PPARα and TTNPB for RARα) leads to at least an additive, and often a synergistic enhancement of the activation pathway of the other IR of the heterodimer pair (e.g. the PPARα pathway). See e.g., WO 94/15902, published Jul. 21, 1994; R. Mukherjee et al., J. Steroid Biochem. Molec. Biol., 51:157-166 (1994); and L. Jow and R. Mukherjee, J. Biol. Chem., 270:3836-40 (1995).

RXR agonists compounds which have been identified so far have exhibited significant therapeutic utility, but they have also exhibited some undesirable side effects, such as elevation of triglycerides and suppression of the thyroid hormone axis (see, e.g., Sherman, S. I. et al., N. Engl. J. Med. 340(14):1075-1079 (1999).

### SUMMARY OF THE INVENTION

The present invention is directed to compounds represented by Structural Formula I and geometric isomers, pharmaceutically acceptable salts, solvates and hydrates thereof:

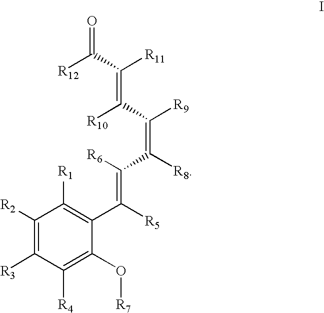

In Structural Formula I, R₁ is H or a halo. R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, a C₂-C₆ alkynyl, a C₂-C₆ haloalkynyl, an aryl, a heteroaryl, a C₁-C₆ alkoxy, an aryloxy, or an amino group represented by the formula NR₁₃R₁₄. R₃ is hydrogen, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, a C₂-C₆ haloalkynyl, an aryl, a heteroaryl, a C₁-C₆ alkoxy, an aryloxy. Alternatively, R₂ and R₃ or R₃ and R₄ taken together with the carbons to which they are attached form an optionally substituted five, six or seven membered carbocyclic or heterocyclic ring. R₅ and R₁₀ are each, independently, methyl, fluoromethyl, difluoromethyl, or trifluoromethyl. R₆, R₈, R₉ and R₁₁ are each, independently, H or F. However, in Structural Formula I, at least one of R₈ or R₉ is F, or at least one of R₅ or R₁₀ is fluoromethyl, difluoromethyl, or trifluoromethyl. R₇ is an optionally substituted C₁-C₆ alkyl, an optionally substituted C₂-C₅ alkenyl, C₁-C₆ haloalkyl, an optionally substituted aryl, or an optionally substituted heteroaryl. R₁₂ is OR₁₅, OCH(R₁₇)OC(O)R₁₆, NR₁₇R₁₈ or an aminoalkyloxy. R₁₃ and R₁₄ are each, independently, H or an C₁-C₆ alkyl or taken together with the nitrogen to which they are attached form a heterocycle. R₁₅ is H, a C₁-C₆ alkyl, an aryl or an aralkyl. R₁₆ is a C₁-C₆ alkyl, an aryl or an aralkyl. R₁₇ and R₁₈ are each, independently, H or a C₁-C₆ alkyl, an aryl or an aralkyl.

In one embodiment, the present invention relates to a method of modulating retinoid X receptor activity in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating RXRα:PPARα heterodimer activity in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating RXRα:PPARγ heterodimer activity in a mammal by administering to the mammal a pharmaceutically effective amount of at least one compound represented by Structural Formula L or a geometric isomer, pharmaceutically acceptable salts, solvates or hydrates thereof.

In another embodiment, the present invention relates to a method of increasing HDL cholesterol levels and reducing triglyceride levels in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating lipid metabolism in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of lowering blood glucose levels without altering serum triglyceride levels in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of treating or preventing a disease or condition in a mammal, wherein the disease or condition are selected from the group consisting of syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness. The method involves administering to the mammal a pharmaceutically effective amount of at least one compound represented by Structural Formula I, or a geometric isomer, pharmaceutically acceptable salts, solvates or hydrates thereof.

In another embodiment, the present invention also relates to pharmaceutical compositions which include a pharmaceutically acceptable carrier and at least one compound represented by Structural Formula I, or a geometric isomer, pharmaceutically acceptable salts, solvates or hydrates thereof.

In yet another embodiment, the present invention relates to a method of making a compound represented by Structural Formula I.

The compounds of the present invention and geometric isomers, pharmaceutically acceptable salts, solvates and hydrates thereof are believed to be effective in treating diseases or conditions that are mediated by retinoid X receptors or heterodimers of retinoid X receptors. Therefore, the compounds of the invention and pharmaceutically acceptable salts, solvates and hydrates thereof are believed to be effective in treating syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness. In addition, the compounds of the invention exhibit fewer side effects than compounds currently used to treat these conditions.

### DETAILED DESCRIPTION OF INVENTION

The term “alkyl”, alone or in combination, means a straight-chain or branched-chain alkyl radical having from 1 to about 10 carbon atoms. Examples of such radical include methyl, ethyl, n-propyl, isopropyl, n-butyl, isobutyl, sec-butyl, tert-butyl, tert-amyl, pentyl, hexyl, heptyl, octyl and the like. Preferably, an alkyl group has from 1 to 6 carbon atoms.

The term “alkenyl”, alone or in combination, means a straight-chain or branched-chain hydrocarbon radical having one or more carbon-carbon double-bonds and having from 2 to about 18 carbon atoms. Examples of alkenyl radicals include ethenyl, propenyl, 1,4-butadienyl and the like. Preferably, an alkenyl group has from 1 to 6 carbon atoms.

The term “alkynyl”, alone or in combination, means a straight-chain or branched-chain hydrocarbon radical having one or more carbon-carbon triple-bonds and having from 2 to about 10 carbon atoms. Examples of alkynyl radicals include ethynyl, propynyl, butynyl and the like. Preferably, an alkynyl group has from 1 to 6 carbon atoms.

The term “aryl”, alone or in combination, means an optionally substituted six-membered carbocyclic aromatic ring systems (e.g. phenyl), fused polycyclic aromatic ring systems (e.g. naphthyl and anthracenyl) and aromatic ring systems fused to carbocyclic non-aromatic ring systems (e.g., 1,2,3,4-tetrahydronaphthyl). Aryl groups include polyaromatic rings and polycyclic ring systems of from two to four, more preferably two to three, and most preferably two rings. Aryl rings typically have from 6 to about 18 carbon atoms.

The term “alkoxy”, alone or in combination, means an alky ether radical wherein the term alkyl is defined as above. Examples of alkoxy radicals include methoxy, ethoxy, ni-propoxy, iso-propoxy, n-butoxy, iso-butoxy, sec-butoxy, tert-butoxy and the like.

The term “aryloxy”, alone or in combination, means an aryl ether radical wherein the term aryl is defined as above. Examples of aryloxy radicals include phenoxy, benyloxy and the like.

The term “cycloalkyl”, alone or in combination, means a saturated monocyclic, bicyclic or tricyclic alkyl radical wherein each cyclic moiety has about 3 to about 8 carbon atoms.

The term “cycloalkenyl”, alone or in combination, means a monocyclic, bicyclic or tricyclic alkyl radical having one or more non-aromatic double bond wherein each cyclic moiety has about 3 to about 8 carbon atoms.

The term “aralkyl”, alone or in combination, means an alkyl radical as defined above in which one hydrogen atom is replaced by an aryl radical as defined above. Examples of aralkyl groups include benzyl, 2-phenylethyl and the like.

The terms “alkyl”, “alkenyl” and “alkynyl” include straight-chain or branched-chain.

The terms “heteroalkyl”, “heteroalkenyl” and “heteroalkynyl” include optionally substituted C₁-C₁₀ alkyl, C₁-C₁₀ alkenyl and C₁-C₁₀ alkynyl structures, as described above, in which one or more skeletal atoms is oxygen, nitrogen, sulfur, or combinations thereof.

The terms “haloalkyl”, “haloalkenyl” and “haloalkynyl” include C₁-C₁₀ alkyl, C₁-C₁₀ alkenyl and C₁-C₁₀ alkynyl structures, as described above, that are substituted with one or more F, Cl, Br or I or with combinations thereof.

The terms “cycloalkyl” and “cycloalkenyl” include optionally substituted C₃-C₅ carbocyclic structures.

The term “carbocyclic” means a cycloalkyl, cycloalkenyl or aryl wherein the cyclic moiety is composed of carbon atoms.

The term “heterocycle” includes optionally substituted, saturated and/or unsaturated, three- to eight-membered cyclic structures wherein the cyclic moiety includes one or more oxygen, nitrogen, sulfur, or combinations thereof.

The term “heteroaryl” refers to optionally substituted five- to eight-membered monocyclic heterocyclic aromatic rings and eight- to eighteen-membered polycyclic fused ring systems having at least one aromatic heterocyclic ring. The heterocyclic rings may contain one or more heteroatoms selected from the group consisting of oxygen, nitrogen and sulfur. Polycyclic heteroaryl ring systems can have from two to four, more preferably two to three, and most preferably two aromatic rings. Examples of heteroaryl groups include, without limitation, furyl, pyrrolyl, pyrrolidinyl, thienyl, pyridyl, piperidyl, indolyl, quinolyl, thiazole, benzthiazole, triazole, benzo[b]furanyl, benzo[b]thienyl, thieno[2,3-c]pyridinyl, benzo[d]isoxazolyl, indazolyl, imidazo[1,2-a]pyridinyl, isoquinolinyl, pyridyl, pyrrolyl, isoxazolyl, and pyrimidinyl.

The substituents of an “optionally substituted” structure may include, but are not limited to, one or more of the following preferred substituents: F, Cl, Br, I, CN, NO₂, NH₂, NHCH₃, N(CH₃)₂, SH, SCH₃, OH, OCH₃, OCF₃, CH₃, CF₃.

The term “halo” includes to F, Cl, Br or I.

An aminoalkyl group is an alkyl group having from one to six carbon atoms which is substituted with at least one amine represented by —NR₂₁R₂₂, in which R₂₁, and R₂₂ are each, independently, a C₁-C₆ alkyl, an aryl or an aralkyl, or R₂₁ and R₂₂ taken together with the nitrogen to which they are attached form a five or six membered heterocycloalkyl.

The term “RXR modulator” refers to a compound that binds to one or more Retinoid X Receptors and modulates (i.e., increases or decreases the transcriptional activity and/or biological properties of the given receptor dimer) the transcriptional activity of an RXR homodimer (i.e., RXR:RXR) and/or RXR in the context of a heterodimer, including but not limited to heterodimer formation with peroxisome proliferator activated receptors (e.g., RXR:PPARα,β,γ1 or γ2), thyroid receptors (e.g., RXR:TRα or β), vitamin D receptors (e.g., RXR:VDR), retinoic acid receptors (e.g., RXR:RARα,β or γ), NGFIB receptors (e.g. RXR:NGFIB), NURR1 receptors (e.g., RXR:NURR1) LXR receptors (e.g., RXR:LXRα,β), DAX receptors (e.g., RXR:DAX), as well as other orphan receptors that form heterodimers with RXR, as either an agonist, partial agonist and/or antagonist. The particular effect of an RXR modulator as an agonist, partial agonist and/or antagonist will depend upon the cellular context as well as the heterodimer partner in which the modulator compounds acts.

In a first embodiment, either R₈ is F or R₁₀ is fluoromethyl, difluoromethyl, or trifluoromethyl in the compounds represented by Structural Formula I, separately or with their respective pharmaceutical compositions.

In a second embodiment, either R₈ is F or R₁₀ is fluoromethyl, difluoromethyl, or trifluoromethyl and R₉ is H and R₅ is methyl in the compounds represented by Structural Formula I, separately or with their respective pharmaceutical compositions.

In a third embodiment, R₈ is F and R₁₀ is methyl in the compounds represented by Structural Formula I and in the first or second embodiment, separately or with their respective pharmaceutical compositions.

In a fourth embodiment, R₈ is hydrogen and R₁₀ is trifluoromethyl in the compounds represented by Structural Formula I and in the first or second embodiment, separately or with their respective pharmaceutical compositions.

In a fifth embodiment, the compounds represented by Structural Formula I or in compounds of the first, second, third or fourth embodiment, separately or with their respective pharmaceutical compositions, have R₅ and R₆ in a cis configuration.

In a sixth embodiment, R₁ and R₃ of are each hydrogen, and R₂ and R₄ are each, independently, a C₁-C₆ alkyl in the compounds represented by Structural Formula I or in compounds of the first, second, third, fourth or fifth embodiment, and their respective pharmaceutical compositions.

In a seventh embodiment, R₁ and R₃ are each hydrogen, and R₂ and R₄ are the same C₁-C₆ alkyl in the compounds represented by Structural Formula I or in compounds of the first, second, third, fourth or fifth embodiment, and their respective pharmaceutical compositions.

In an eighth embodiment, R₁ and R₃ are each hydrogen, and R₂ and R₄ are both iso-propyl or tert-butyl in the compounds represented by Structural Formula I or in compounds of the first, second, third, fourth, or fifth embodiment, and their respective pharmaceutical compositions.

In a ninth embodiment, R₇ is a C₂-C₅ alkyl in the compounds represented by Structural Formula I or compounds of the first, second, third, fourth, fifth, sixth, seventh or eighth embodiment, and their respective pharmaceutical compositions.

In a tenth embodiment, R₇ is a C₂-C₅ alkyl which is substituted with from one to nine fluoro groups in the compounds represented by Structural Formula I or compounds of the first, second, third, fourth, fifth, sixth, seventh or eighth embodiment, and their respective pharmaceutical compositions.

In an eleventh embodiment, R₅ and R₆ are in a cis configuration, R₇ is a C₂-C₅ alkyl which is optionally substituted with from one to nine fluoro groups, and R₁₂ is OH in the compounds represented by Structural Formula I or compounds of the first, second, third or fourth embodiment, and their respective pharmaceutical compositions.

In an twelfth another embodiment, R₅ and R₆ are in a cis configuration, R₁ and R₃ are both hydrogen, R₂ and R₄ are both isopropyl or both isobutyl, R₇ is a C₂-C₅ alkyl which is optionally substituted with from one to nine fluoro groups, and R₁₂ is OH in the compounds represented by Structural Formula I or compounds of the first, second, third or fourth embodiment, and their respective pharmaceutical compositions.

Preferably, R₁ in Structural Formula I and in embodiments 1-12 is hydrogen.

Preferably, R₂ in Structural Formula I and in embodiments 1-12 is an optionally substituted C₁-C₆ alkyl, C₁-C₆ haloalkyl, an optionally substituted C₃-C₆ cycloalkyl, aryl, and heteroaryl. Most preferrably R₂ is optionally substituted C₁-C₆ alkyl.

Preferably, R₃ in Structural Formula I and in embodiments 1-12 is hydrogen, optionally substituted C₁-C₅ alkyl and heteroalkyl. More preferrably, R₃ is hydrogen.

Preferably, R₄ in Structural Formula I and in embodiments 1-12 is optionally substituted C₁-C₆ alkyl, C₁-C₆ haloalkyl, optionally substituted C₃-C₆ cycloalkyl, aryl, and heteroaryl. More preferrably R₄ is optionally substituted C₁-C₆ alkyl.

Preferred groups for R₅ and R₁₀ in Structural Formula I and in embodiments 1-12 are each, independently, methyl or trifluoromethyl.

Preferred R₇ groups in Structural Formula I and in embodiments 1-12 include optionally substituted C₂-C₅ alkyl or C₂-C₅ haloalkyl. More preferrably, R₇ is C₂-C₅ alkyl or a C₂-C₅ alkyl which is substituted with from one to three fluoro groups.

R₈ is preferably F in Structural Formula I and in embodiments 1-12.

Preferably, R₁₂ is OH in Structural Formula I and in embodiments 1-12.

Compounds of the present invention include, but are not limited to, the following group of compounds:

7-[3,5-di-tert-butyl-2-(2,2-difluoroethoxy)-phenyl]4-fluoro-3-methyl-octa-2,4,6-trienoic acid;

7-[3,5-di-tert-butyl-2-(2,2-difluoroethoxy)-phenyl]-5-fluoro-3-methyl-octa-2,4,6-trienoic acid;

(2Z,4E,6Z)-7-(2-butoxy-3,5-diisopropylphenyl)-3-trifluoromethyl-octa-2,4,6-trienoic acid;

(2E,4E,6Z)-7-(2-butoxy-3,5-diisopropylphenyl)-3-trifluoromethyl-octa-2,4,6-trienoic acid;

(2E,4E,6E)-3-methyl-7-(2-ethoxy-3,5-di-tert-butylphenyl)-8,8,8-trifluoroocta-2,4,6-trienoic acid;

and pharmaceutically acceptable salts, solvates and hydrates thereof.

The compounds of Formula I represent a select group of compounds among previously disclosed RXR modulators that have insulin sensitizing activity, but do not suppress the thyroid axis and do not elevate triglycerides. These compounds are heterodimer selective modulators of RXR activity. They bind to RXR with high affinity (generally Kᵢ<50 nM) and produce potent synergistic activation of the RXR:PPARγ heterodimer, but preferably do not synergize with RAR agonists at the RXR:RAR heterodimer. This synergistic activation of PPARγ in vitro is contemplated to be a major determinant of the antidiabetic efficacy of the compounds in vivo. In addition, the compounds of the present invention have reduced susceptibility to oxidative metabolism relative to previously disclosed RXR modulators.

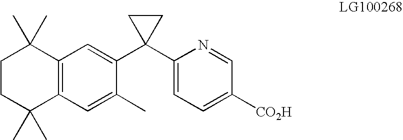

Compounds, such as LG100268, that are fall RXR homodimer agonists are efficacious insulin sensitizers in rodent models of Type II Diabetes, but they also raise triglycerides and suppress the thyroid hormone axis.

The compounds of the invention are heterodimer selective modulators of RXR activity. Those compounds that have a carbon chain length at the R⁷ position and appropriate substituents at R¹, R², R³, and R⁴ within the scope of the present invention maintain the desirable insulin sensitizing activity and eliminate or reduce both the suppression of the thyroid axis and triglyceride elevations.

The compounds of the invention are expected to be efficacious insulin sensitizers and to eliminate undesirable increases in triglycerides and suppression of T4 because they selectively bind to RXR but do not significantly activate the RXR:RAR heterodimer.

When administered to obese, insulin resistant db/db mice (100 mg/kg by daily oral gavage for 14 days) these heterodimer selective RXR modulators are expected to lower both plasma glucose and triglycerides. However, unlike either full agonists (e.g., LG100268) or partial agonists that exhibit less than 50% activity at the RXR:RAR heterodimer, they are not expected to suppress total circulating levels of T4, or increase triglycerides.

When administered to transgenic mice carrying the human apo A-I gene the compounds of the invention are expected to increase HDL cholesterol, but unlike LG100268 they are not expected to raise triglycerides. These effects are consistent with activation of PPARα, and the compounds of the invention are expected to synergize with PPARα agonists.

The compounds of the present invention possess particular application as RXR modulators and in particular as dimer-selective RXR modulators including, but not limited to, RXR homodimer antagonists, and agonists, partial agonists and antagonists of RXRs in the context of a heterodimer.

In a second aspect, the present invention provides a method of modulating processes mediated by RXR homodimers and/or RXR heterodimers comprising administering to a patient an effective amount of a compound of the invention as set forth above. The compounds of the present invention also include all pharmaceutically acceptable salts, as well as esters and amides. As used in this disclosure, pharmaceutically acceptable salts include, but are not limited to: pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydoxymethyl) aminomethane. Additional pharmaceutically acceptable salts are known to those skilled in the art.

The compounds of the present invention are useful in the modulation of transcriptional activity through RXR in the context of heterodimers other than RXR:RARα,β,γ (e.g., RXR:PPARα,β,γ; RXR:TR; RXR:VDR; RXR:NGFIB; RXR:NURR1; RXR:LXRα,β, RXR:DAX), including any other intracellular receptors (IRs) that form a heterodimer with RXR. For example, application of the compounds of the present invention to modulate a RXRα:PPARα heterodimer is useful to modulate, i.e. increase, HDL cholesterol levels and reduce triglyceride levels. Yet, application of many of the same compounds of the present invention to a RXRα:PPARγ heterodimer modulates a distinct activity, i.e., modulation of adipocyte biology, including effects on the differentiation and apoptosis of adipocytes, which will have implications in the treatment and/or prevention of diabetes and obesity. In addition, use of the modulator compounds of the present invention with activators of the other heterodimer partner (e.g., fibrates for PPARα and thiazolidinediones for PPARγ) can lead to a synergistic enhancement of the desired response. Likewise, application of the modulator compounds of the present invention in the context of a RXRα:VDR heterodimer will be useful to modulate skin related processes (e.g., photoaging, acne, psoriasis), malignant and pre-malignant conditions and programmed cell death (apoptosis). Further, it will be understood by those skilled in the art that the modulator compounds of the present invention will also prove useful in the modulation of other heteromer interactions that include RXR, e.g., trimers, tetramers and the like.

In the context of an RXR homodimer, the compounds of the present invention function as partial agonists. Further, when the modulator compounds of the present invention are combined with a corresponding modulator of the other heterodimeric partner, a surprising synergistic enhancement of the activation of the heterodimer pathway can occur. For example, with respect to a RXRα:PPARα heterodimer, the combination of a compound of the present invention with clofibric acid or gemfibrozil unexpectedly leads to a greater than additive (i.e. synergistic) activation of PPARα responsive genes, which in turn is useful to modulate serum cholesterol and triglyceride levels and other conditions associated with lipid metabolism.

Whether acting on an RXR heterodimer pathway, or the RXR homodimer pathway, it will also be understood by those skilled in the art that the dimer-selective RXR modulator compounds of the present invention will prove useful in any therapy in which agonists, partial agonists and/or full antagonists of such pathways will find application. Importantly, because the compounds of the present invention can differentially activate RXR homodimers and RXR heterodimers, their effects will be tissue and/or cell type specific, depending upon the cellular context of the different tissue types in a given patient. For example, compounds of the present invention will exert an RXR antagonist effect in tissues where RXR homodimers prevail, and partial agonist or full agonist activity on the PPAR pathway where RXRα:PPARα heterodimers prevail (e.g., in liver tissue). Thus, the compounds of the present invention will exert a differential effect in various tissues in an analogous fashion to the manner in which various classes of estrogens and antiestrogens (e.g., Estrogen, Tamoxifen, Raloxifen) exert differential effects in different tissue and/or cell types (e.g., bone, breast, uterus). See e.g., M. T. Tzukerman et al., Mol. Endo, 8:21-30 (1994); D. P. McDonnell et al., Mol. Endo., 9:659-669 (1995). However, in the present case, it is believed that the differential effects of the compounds of the present invention are based upon the particular dimer pair through which the compound acts, rather than through different transactiving regions of the estrogen receptor in the case of estrogens and antiestrogens. However, it is possible that they also function, in part, by tissue selectivity.

The particular conditions that may be treated with the compounds of the present invention include, but are not limited to, skin-related diseases, such as actinic keratoses, arsenic keratoses, inflammatory and non-inflammatory acne, psoriasis, ichthyoses and other keratinization and hyperproliferative disorders of the skin, eczema, atopic dermatitis, Darriers disease, lichen planus, prevention and reversal of glucocorticoid damage (steroid atrophy), as a topical anti-microbial, as skin pigmentation agents and to treat and reverse the effects of age and photo damage to the skin. With respect to the modulation of malignant and pre-malignant conditions, the compounds may also prove useful for the prevention and treatment of cancerous and pre-cancerous conditions, including, premalignant and malignant hyperproliferative diseases and cancers of epithelial origin such as cancers of the breast, skin, prostate, cervix, uterus, colon, bladder, esophagus, stomach, lung, larynx, oral cavity, blood and lymphatic system, metaplasias, dysplasias, neoplasias, leukoplakias and papillomas of the mucous mem-branes and in the treatment of Kaposis sarcoma. In addition, the present compounds may be used as agents to treat and prevent various cardiovascular diseases, including, without limitation, diseases associated with lipid metabolism such as dyslipidemias, prevention of restenosis and as an agent to increase the level of circulating tissue plasminogen activator (TPA), metabolic diseases such as obesity and diabetes (i.e., non-insulin dependent diabetes mellitus and insulin dependent diabetes mellitus), the modulation of differentiation and proliferation disorders, as well as the prevention and treatment of neurodegenerative diseases such as Alzheimer's disease, Parkinson's disease and Amyotrophic Lateral Sclerosis (ALS), and in the modulation of apoptosis, including both the induction of apoptosis and inhibition of T-Cell activated apoptosis.

Furthermore, it will be understood by those skilled in the art that the compounds of the present invention, including pharmaceutical compositions and formulations containing these compounds, can be used in a wide variety of combination therapies to treat the conditions and diseases described above. Thus, the compounds of the present invention can be used in combination with modulators of the other heterodimeric partner with RXR (i.e., in combination with PPARα modulators, such as fibrates, in the treatment of cardiovascular disease, and in combination with PPARγ modulators, such thiazolidinediones, in the treatment of diabetes, including non-insulin dependent diabetes mellitus and insulin dependent diabetes mellitus, and with agents used to treat obesity) and with other therapies, including, without limitation, chemotherapeutic agents such as cytostatic and cytotoxic agents, immunological modifiers such as interferons, interleukins, growth hormones and other cytokines, hormone therapies, surgery and radiation therapy.

By utilizing the compounds of the present invention with modulators of the other heterodimeric partner one is able to utilize lower dosages of either or both modulators, thereby leading to a significant decrease in the side-effects associated with such modulators when employed alone at the strengths required to achieve the desired effect. Thus, the modulator compounds of the present invention, when utilized in combination therapies, provide an enhanced therapeutic index (i.e., significantly enhanced efficacy and/or decrease side-effect profiles) over utilization of the compounds by themselves.

Prodrugs are compounds of the present invention, which have chemically or metabolically cleavable groups and become by solvolysis or under physiological conditions the compounds of the invention which are pharmaceutically active in vivo. Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Particularly preferred esters as prodrugs are methyl, ethyl, propyl, isopropyl, n-butyl, isobutyl, tert-butyl, morpholinoethyl, and N,N-diethylglycolamido.

Methyl ester prodrugs may be prepared by reaction of the acid form of a compound of formula I in a medium such as methanol with an acid or base esterification catalyst (e.g., NaOH, H₂SO₄). Ethyl ester prodrugs are prepared in similar fashion using ethanol in place of methanol.

Morpholinylethyl ester prodrugs may be prepared by reaction of the sodium salt of a compound of Structural Formula I (in a medium such as dimethylformamide) with 4(2-chloroethyl)morphine hydrochloride (available from Aldrich Chemical Co., Milwaukee, Wis. USA, Item No. C4,220-3).

The term “pharmaceutically acceptable” means that the carrier, diluent, excipients and salt must be compatible with the other ingredients of the formulation, and not deleterious to the recipient thereof. Pharmaceutical formulations of the present invention are prepared by procedures known in the art using well known and readily available ingredients.

“Preventing” refers to reducing the likelihood that the recipient will incur or develop any of the pathological conditions described herein.

By virtue of its acidic moiety, a compound of Structural Formula I forms salts with pharmaceutically acceptable bases. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts, zinc salts, and ammonium salts, as well as salts made from physiologically acceptable organic bases such as methylamine, dimethylamine, trimethylamine, ethylamine, diethylamine, triethylamine, morpholine, pyridine, piperidine, piperazine, picoline, nicotinamide, urea, tris(hydroxymethyl)aminomethane, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-p-phenethylamine, dehydroabietylamine, N,N′-bisdehydroabietylamine, glucamine, N-methylglucamine, collidine, quinine, quinoline, and basic amino acid such as lysine and arginine. These salts may be prepared by methods known to those skilled in the art.

Compounds of Structural Formula I, which are substituted with a basic group, may exist as salts with pharmaceutically acceptable acids. The present invention includes such salts. Examples of such salts include hydrochlorides, hydrobromides, sulfates, methanesulfonates, nitrates, maleates, acetates, citrates, cinnamates, picrate, formate, fumarates, tartrates [e.g. (+)-tartrates, (−)-tartrates or mixtures thereof including racemic mixtures], succinates, benzoates and salts with amino acids such as glutamic acid.

Certain compounds of Structural Formula I and their salts may also exist in the form of solvates, for example hydrates, and the present invention includes each solvate and mixtures thereof.

Certain compounds of Structural Formula I may exist in different tautomeric forms or as different geometric isomers, and the present invention includes each tautomer and/or geometric isomer of compounds of Structural Formula I and mixtures thereof

Certain compounds of Structural Formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Structural Formula I and mixtures thereof.

Certain compounds of Structural Formula I may exist in zwitterionic form and the present invention includes each zwitterionic form of compounds of Structural Formula I and mixtures thereof.

Certain compounds of Structural Formula I and their salts may exist in more than one crystal form. Polymorphs of compounds represented by Structural Formula I form part of this invention and may be prepared by crystallization of a compound of Structural Formula I under different conditions. For example, using different solvents or different solvent mixtures for recrystallization; crystallization at different temperatures; various modes of cooling, ranging from very fast to very slow cooling during crystallization. Polymorphs may also be obtained by heating or melting a compound of Structural Formula I followed by gradual or fast cooling. The presence of polymorphs may be determined by solid probe nmr spectroscopy, ir spectroscopy, differential scanning calorimetry, powder X-ray diffraction or such other techniques.

The language a “therapeutically effective amount” or “pharmaceutically effective amount” is intended to include an amount which is sufficient to mediate a disease or condition and prevent its further progression or ameliorate the symptoms associated with the disease or condition. Such an amount can be administered prophylactically to a patient thought to be susceptible to development of a disease or condition. Such amount when administered prophylactically to a patient can also be effective to prevent or lessen the severity of the mediated condition. Such an amount is intended to include an amount which is sufficient to modulate one or more retinoid X receptor, such as RXR α, RXR β, and/or RXR γ, which mediates a disease or condition. Conditions mediated by retinoid X receptors include diabetes, dermatologic diseases, inflammatory diseases, neurodegenerative diseases, obesity, cardiovascular diseases, cancer and other proliferative diseases, such as atherosclerosis, uterine leiomyomata. In addition, RXR modulators can be used to promote wound healing or to stimulate hair growth.

The compounds of Structural Formula I, and the pharmaceutically acceptable salts, solvates and hydrates thereof, have valuable pharmacological properties and can be used in pharmaceutical preparations containing the compound or pharmaceutically acceptable salts, esters or prodrugs thereof, in combination with a pharmaceutically acceptable carrier or diluent. They are useful as therapeutic substances in preventing or treating diabetes, dermatologic diseases, inflammatory diseases, neurodegenerative diseases, obesity, cardiovascular diseases, cancer, atherosclerosis, uterine leiomyomata, wounds or hair loss in human or non-human animals. Suitable pharmaceutically acceptable carriers include inert solid fillers or diluents and sterile aqueous or organic solutions. The active compound will be present in such pharmaceutical compositions in amounts sufficient to provide the desired dosage amount in the range described herein.

For oral administration, the compound or salts thereof can be combined with a suitable solid or liquid carrier or diluent to form capsules, tablets, pills, powders, syrups, solutions, suspensions and the like.

The tablets, pills, capsules, and the like may also contain a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. Such compositions and preparations should contain at least 0.1 percent of active compound. The percentage of active compound in these compositions may, of course, be varied and may conveniently be between about 2 percent to about 60 percent of the weight of the unit The amount of active compound in such therapeutically useful compositions is such that an effective dosage will be obtained.

The active compounds can also be administered intranasally as, for example, liquid drops or spray.

For parental administration the compounds of the present invention, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that each syringability exists. It must be stable under the conditions of manufacture and storage and must be preserved against any contamination. The carrier can be solvent or dispersion medium containing, for example, water, ethanol, polyol (e.g. glycerol, propylene glycol and liquid polyethylene glycol), propylene glycol and liquid polyethylene glycol), suitable mixtures thereof, and vegetable oils. The injectable solutions prepared in this manner can then be administered intravenously, intraperitoneally, subcutaneously, or intramuscularly, with intramuscular administration being preferred in humans.

The effective dosage of active ingredient employed may vary depending on the particular compound employed, the mode of administration, the condition being treated and the severity of the condition being treated.

Preferably compounds of the invention or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I or salts thereof) in a unit dose of composition is a therapeutically effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Pharmaceutical formulations of the invention are prepared by combining (e.g., mixing) a therapeutically effective amount of a compound of the invention together with a pharmaceutically acceptable carrier or diluent. The present pharmaceutical formulations are prepared by known procedures using well known and readily available ingredients.

In making the compositions of the present invention, the active ingredient will usually be admixed with a carrier, or diluted by a carrier, or enclosed within a carrier which may be in the form of a capsule, sachet, paper or other container. When the carrier serves as a diluent, it may be a solid, lyophilized solid or paste, semi-solid, or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound. The compounds of the present invention are preferably formulated prior to administration.

For the pharmaceutical formulations any suitable carrier known in the art can be used. In such a formulation, the carrier may be a solid, liquid, or mixture of a solid and a liquid. For example, for intravenous injection the compounds of the invention may be dissolved in at a concentration of about 0.05 to about 5.0 mg/ml in a 4% dextrose/0.5% Na citrate aqueous solution.

Solid form formulations include powders, tablets and capsules. A solid carrier can be one or more substance which may also act as flavoring agents, lubricants, solubilisers, suspending agents, binders, tablet disintegrating agents and encapsulating material.

Tablets for oral administration may contain suitable excipients such as calcium carbonate, sodium carbonate, lactose, calcium phosphate, together with disintegrating agents, such as maize, starch, or alginic acid, and/or binding agents, for example, gelatin or acacia, and lubricating agents such as magnesium stearate, stearic acid, or talc.

In powders the carrier is a finely divided solid which is in admixture with the finely divided active ingredient In tablets the active ingredient is mixed with a carrier having the necessary binding properties in suitable proportions and compacted in the shape and size desired.

Advantageously, compositions containing the compound of Structural Formula I or the salts thereof may be provided in dosage unit form, preferably each dosage unit containing from about 1 to about 500 mg be administered although it will, of course, readily be understood that the amount of the compound or compounds of Structural Formula I actually to be administered will be determined by a physician, in the light of all the relevant circumstances.

Powders and tablets preferably contain from about 1 to about 99 weight percent of the active ingredient which is the novel compound of this invention. Suitable solid carriers are magnesium carbonate, magnesium stearate, talc, sugar, lactose, pectin, dextrin, starch, gelatin, tragacanth, methyl cellulose, sodium carboxymethyl cellulose, low melting waxes, and cocoa butter.

The following pharmaceutical formulations 1 through 8 are illustrative only and are not intended to limit the scope of the invention in any way. “Active Ingredient”, refers to a compound according to Structural Formula I or salts thereof.

Formulation 1

| Hard gelatin capsules are | |
|---|---|
| prepared using the | Quantity |
| following ingredients: | (mg/capsule) |
| Active Ingredient | 250 |
| Starch, dried | 200 |
| Magnesium stearate | 10 |
| Total | 460 mg |

Formulation 2

| A tablet is prepared | |
|---|---|
| using the | Quantity |
| ingredients below: | (mg/tablet) |
| Active Ingredient | 250 |
| Cellulose, microcrystalline | 400 |
| Silicon dioxide, fumed | 10 |
| Stearic acid | 5 |
| Total | 665 mg |

The components are blended and compressed to form
tablets each weighing 665 mg

Formulation 3

| An aerosol solution | |
|---|---|
| is prepared | |
| containing the | |
| following components: | Weight |
| Active Ingredient | 0.25 |
| Ethanol | 25.75 |
| Propellant 22 (Chlorodifluoromethane) | 74.00 |
| Total | 100.00 |

The Active Ingredient is mixed with ethanol and the mixture added to a portion of the propellant 22, cooled to 30° C. and transferred to a filling device. The required amount is then fed to a stainless steel container and diluted with the remainder of the propellant. The valve units are then fitted to the container.

Formulation 4
Tablets, each containing 60 mg of Active ingredient,
are made as follows:

| | |
|---|---|
| Active Ingredient | 60 mg |
| Starch | 45 mg |
| Microcrystalline cellulose | 35 mg |
| Polyvinylpyrrolidone (as 10% solution in water) | 4 mg |
| Sodium carboxymethyl starch | 4.5 mg |
| Magnesium stearate | 0.5 mg |
| Talc | 1 mg |
| Total | 150 mg |

The Active Ingredient, starch and cellulose are passed through a No. 45 mesh U.S. sieve and mixed thoroughly. The aqueous solution containing polyvinylpyrrolidone lo is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets 15 each weighing 150 mg.

Formulation 5
Capsules, each containing 80 mg of Active Ingredient,
are made as follows:

| | |
|---|---|
| Active Ingredient | 80 mg |
| Starch | 59 mg |
| Microcrystalline cellulose | 59 mg |
| Magnesium stearate | 2 mg |
| Total | 200 mg |

The Active Ingredient, cellulose, starch, and magnesium stearate are blended, passed through a No. 45 mesh U.S. sieve, and filled into hard gelatin capsules in 200 mg quantities.

### Formulation 6

Suppositories, each containing 225 mg of Active Ingredient, are made as follows:

| | |
|---|---|
| Active Ingredient | 225 mg |
| Saturated fatty acid glycerides | 2.000 mg |
| Total | 2.225 mg |

The Active Ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

Formulation 7
Suspensions, each containing 50 mg of Active
Ingredient per 5 ml dose, are made as follows:

| | |
|---|---|
| Active Ingredient | 50 mg |
| Sodium carboxymethyl cellulose | 50 mg |
| Syrup | 1.25 ml |
| Benzoic acid solution | 0.10 ml |
| Flavor | q.v. |
| Color | q.v. |
| Purified water to total | 5 ml |

The Active Ingredient is passed through a No. 45 mesh U.S. sieve and mixed with the sodium carboxymethyl cellulose and syrup to form a smooth paste. The benzoic acid solution, flavor and color are diluted with a portion of the water and added, with stirring. Sufficient water is then added to produce the required volume.

Formulation 8
An intravenous formulation may be prepared as follows:

| | |
|---|---|
| Active Ingredient | 100 mg |
| Isotonic saline | 1,000 ml |

The solution of the above materials generally is administered intravenously to a subject at a rate of 1 ml per minute.

Synthesis

The compounds of the invention can be prepared by reacting a substituted (2-iodo-1-methylvinyl) benzene (VII) and a substituted 5-tributylstannanyl-penta-2,4-dienoic acid alkyl ester (see Scheme III). The substituted (2-iodo-1-methylvinyl) benzene (VII) is prepared from a substituted iodobenzene (II) (see Scheme I). The substituted iodobenzene (II) is dissolved in a solvent and treated with a catalytic amount of copper iodide and dichlorobis(triphenylphosphine)palladium(II) or tetrakistriphenylphosphinepalladium(0) (typically about 0.05 eq. to about 0.15 eq. of each) and excess aprotic base (typically about 2 eq. to about 10 eq.). After about 5 min. to about 30 min., about 1 eq. to about 3 eq. of trimethylsilyl acetylene (III) is added, and the reaction is heated in a sealed tube to about 50° C. to about 120° C. for about 8 hrs. to about 16 hrs. to form a (substituted phenyl)-trimethylsilyl acetylene (IV).

The (substituted phenyl)-trimethylsilyl acetylene (IV) is dissolved in a solvent and treated with about 0.1 eq. to about 0.5 eq. of nickel(II) acetylacetonate (Ni(acac)₂) and about 3 eq. to about 8 eq. of dimethyl zinc (V) which is optionally substituted with from one to six fluoro groups. After about 8 h to about 20 h, a [2-(substituted phenyl)-propen-1-yl]-trimethylsilane (VI) is formed.

A solution of [2-(substituted phenyl)-propen-1-yl]-trimethylsilane (VI) in a nonpolar solvent is cooled to about 10° C. to about −20° C., then about 1 eq. to bout 2 eq. of iodine monochloride is added. After about 1 h to about 4 h, a substituted (2-iodo-1-methylvinyl) benzene (VII) is formed.

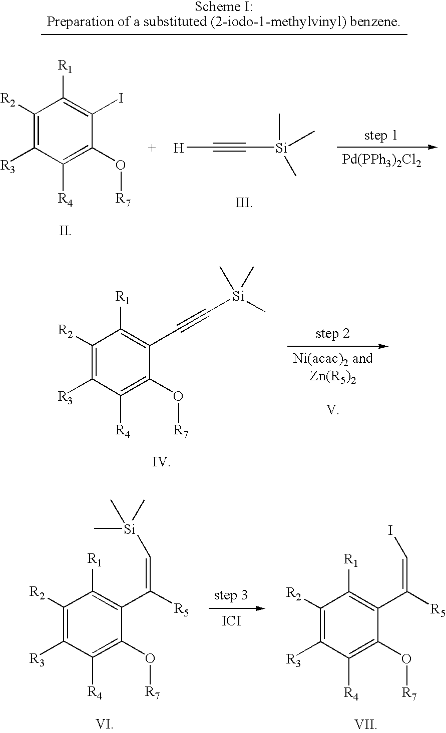

The substituted 5-tributylstannanyl-penta-2,4-dienoic acid alkyl ester (XIII) can be prepared from an optionally substituted alkyl 3-methyl4-oxocrotonate (XI) (see Scheme II). In the first step, dialkylchlorophosphate (IX) and lithium hexamethyldisilazane (LiHMDS) are added to a solution of methyl phenyl sulfone (VIII) that is optionally substituted with a fluoro group in an aprotic solvent, preferably an ether, that has been cooled to about −50° C. to about −100° C. After about 15 min. to about 1 hr., the alkyl 3-methyl4-oxocrotonate (XI) is added, and the reaction is allowed to warm to room temperature and is stirred for about 8 hrs. to about 20 hrs. to form an optionally substituted 5-benzenesulfonyl-3-methyl-penta-2,4-dienoic acid alkyl ester (XII). About 1.5 eq. to 2.5 eq. of the methyl phenyl sulfone (VIII), about 1.5 eq. to about 2.5 eq. of the dialkylchlorophosphate (IX), and about 3.0 eq. to about 5 eq. of the lithium hexamethyldisilazane with respect to the alkyl 3-methyl4-oxocrotonate (XI) are typically present in the reaction mixture.

A mixture of the 5-benzenesulfonyl-3-methyl-penta-2,4-dienoic acid alkyl ester (XII), about 1.5 eq. to about 3 eq. of tributyl tin hydride (SnBu₃H) and a catalytic amount of a free radical initiator such as 2,2′-azobisisobutyronitrile (AIBN) in an organic solvent is heated to about 50° C. to about 120° C. for about 8 hrs. to about 20 hrs. to form an optionally substituted 3-methyl-5-tributylstannayl-penta-2,4-dienoic acid alkyl ester (XIII).

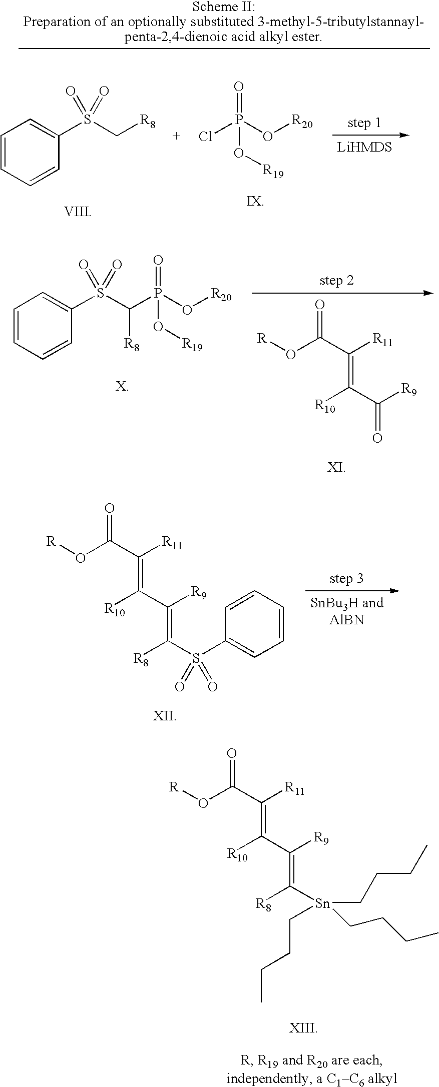

The substituted (2-iodo-1-methylvinyl) benzene (VII) and the 3-methyl-5-tributylstannayl-penta-2,4-dienoic acid alkyl ester (XIII) (about 1 eq. to about 1.5 eq.) are combined in an organic solvent with a catalytic amount (about 0.05 eq. to about 0.15 eq.) of dichlorobis(triphenylphosphine)palladium(II). The reaction is heated to about 50° C. to about 100° C. for about 1 h to about 4 h to form a 3-methyl-7-(substituted phenyl)-octa-2,4,6-trienoic acid alkyl ester (XIV). A 3-methyl-7-(substituted phenyl)-octa-2,4,6-trienoic acid (XV) can be formed by treating the 3-methyl-7-substituted phenyl)-octa-2,4,6-trienoic acid alkyl ester (XIV) with an alkali metal hydroxide (see Scheme III).

Example 2 was prepared using the methods of Schemes I, II, and III.

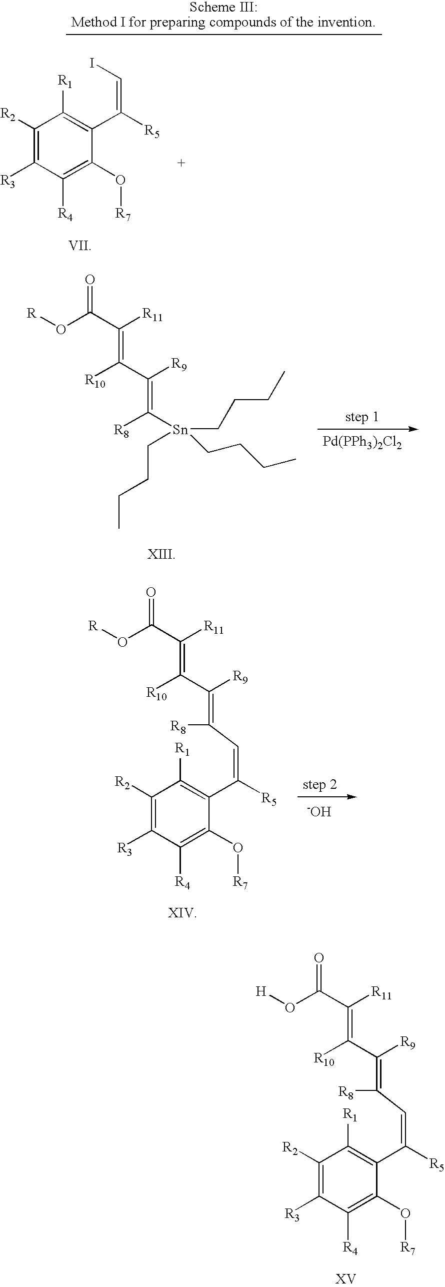

Alternatively, compounds of the invention can be prepared by a second method from a phenyl substituted with α,β-unsaturated carbonyl (XVI) (see Scheme IV). In this method, compound X is prepared via the method of Scheme II, step 1. A phenyl substituted with α,β-unsaturated carbonyl (XVI) is added to a solution of an anion of compound X in an aprotic solvent maintained at about −50° C. to about −100° C. The anion of compound X is prepared by adding lithium hexamethyldisilyazane to a cold solution of compound X in an aprotic solvent. The reaction is allowed to warm to room temperature and is stirred for about 8 h to about 20 h to form an optionally substituted 1-benzenesulfonyl-4-(substituted phenyl)-penta-2,4-diene (XVII). About 1.5 to 2.5 eq. of the methyl phenyl sulfone (VIII) which is optionally substituted with a fluoro group, about 1.5 eq. to about 2.5 eq. of the dialkylchlorophosphate (IX), and about 3.0 eq. to about 5 eq. of the lithium hexamethyldisilazane with respect to compound XVI are typically present in the reaction mixture.

A mixture of the 1-benzenesulfonyl-4-(substituted phenyl)-penta-2,4-diene (XVII), about 1.5 eq. to about 3 eq. of tributyl tin hydride (SnBu₃H) and a catalytic amount of a free radical initiator, such as AIBN, in an organic solvent is heated to about 50° C. to about 120° C. for about 8 h to about 20 h to form an optionally substituted 1-tributylstannayl-4-(substituted phenyl)-penta-1,3diene (XVIII).

A mixture of the 1-tributylstannyl-4-(substituted phenyl)-penta-1,3-diene (XVIII), about 1 eq. to about 2 eq. of an optionally substituted 3-iodo-pro-2-enoic acid (XIX) and about 0.05 eq. to about 0.15 eq. of dichlorobis(triphenylphosphine)-palladium(II) (also referred to herein al “Pd(PPh₃)₂Cl₂”) was heated to about 50° C. to about 100° C. for about 1 h to about 4 h. The reaction is then poured into a potassium fluoride solution and stirred at room temperature for about 0.5 brs. to about 2 hrs. to form a 3-methyl-7-(substituted phenyl)-octa-2,4,6-trienoic acid (XX).

Example 1 was prepared using the method of Scheme IV.

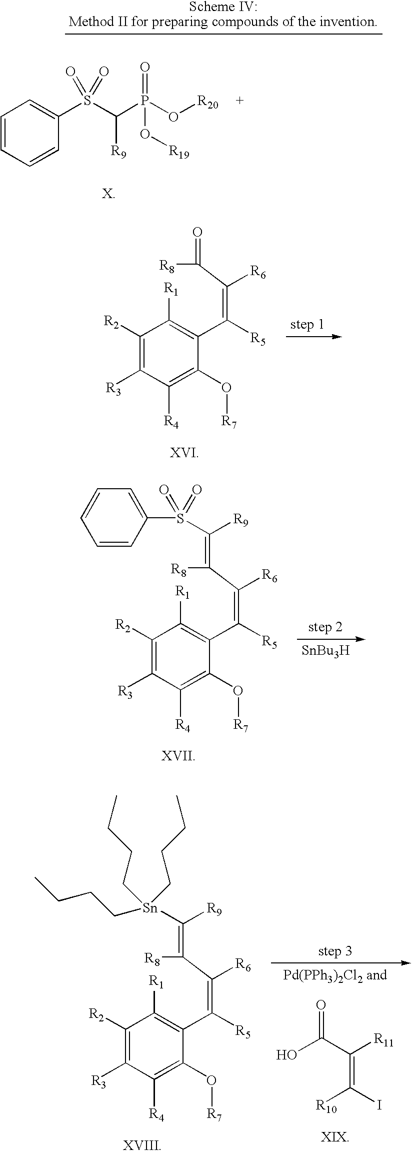

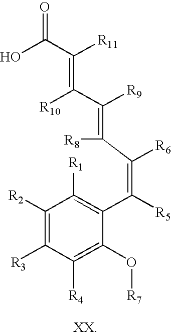

Compounds of the invention can be synthesized by a third method in which a phenyl substituted with an α,β-unsaturated carbonyl (XVI) undergoes an aldol condensation with a ketone (XXI) followed by an elimination reaction to form an optionally substituted 6-(substituted phenyl)-hepta-3,5-dien-2-one (XXI). The reaction is carried out in a basic solvent such as piperidine or pyridine in the presence of about 1 eq. to about 1.5 eq. of an acid. The ketone (XXI) is typically present in a large excess. The 6-(substituted phenyl)-hepta-3,5-dien-2-one (XXII) forms after stirring the reaction mixture for about 0.5 h to about 2 h at room temperature.

A solution of an optionally substituted trialkyl phosphonoacetate (XXIII) in an aprotic solvent is treated with about 1 eq. to about 1.5 eq. of sodium hydride at room temperature. After about 0.5 hrs. to about 1.5 hrs., about 0.5 eq. to about 1 eq. of the 6-(substituted phenyl)-hepta-3,5-dien-2-one (is added to a solution, and the reaction is stirred for about 8 h to about 20 h to form 3-methyl-7-(substituted phenyl)-octa-2,4,6-trienoic acid alkyl ester (XXIV) (see Scheme V). A 3-methyl-7-(substituted phenyl)-octa-2,4,6-trienoic acid (XX) can be formed by treating the 3-methyl-7-(substituted phenyl)-octa-2,4,6-trienoic acid alkyl ester (XXIV) with an alkali metal hydroxide as in Scheme III, step 2.

Examples 3 and 4 were prepared using the method of Scheme V.

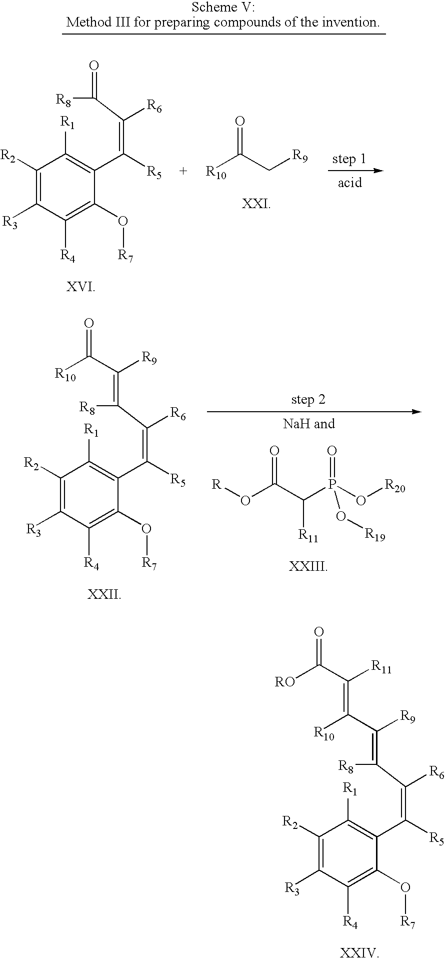

Alternatively, compounds of the invention can be prepared by reacting a phenyl substituted with an α,β-unsaturated carbonyl (XVI) with an anion of a trialkyphosphonoacetate (XXXIX) (see Scheme VI). In this method, a solution of trialkyl phosphonoacetate (XXXI) in an aprotic solvent at about −25° C. to about 10° C. is treated with about 1 eq. to about 1.5 eq. of sodium hydride. After about 0.5 h to about 1.5 h, the phenyl substituted with an α,β-unsaturated carbonyl (XVI) is added and the mixture is stirred for about 4 h to about 24 h to form an optionally substituted 5-(substituted phenyl)-hexa-2,4-dienoic acid alkyl ester (XL).

The 5-(substituted phenyl)-hexa-2,4-dienoic acid alkyl ester (XL) is treated with a reducing agent, such as sodium borohydride, lithium aluminum hydride or diisobutylaluminum hydride, to form an optionally substituted 5-(substituted phenyl)-hexa-2,4-dien-1-ol (XLI). The reaction is typically carried out in a polar solvent at about −25° C. to about 10° C. About 1 eq. to about 5 eq. of the reducing agent is used with respect to the 5-(substituted phenyl)-hexa-2,4-dienoic acid alkyl ester (XL). Typically, the reaction is followed by thin layer chromatography (TLC) to determine when the reaction is complete.

The allylic hydroxy group of 5-(substituted phenyl)-hexa-2,4-dien-1-ol (XLI) is converted to an aldehyde to form an optionally substituted 5-(substituted phenyl)-hexa-2,4-dien-1-al (XLII) by treatment with about 1 eq. to about 2 eq. of 4-methylmorpholine N-oxide (hereinafter “NMO”) and a cataylic amount of tetrapropylammonium perruthenate (hereinafter “TPAP”) (about 0.01 eq. to about 0.1 eq.). The reaction is carried out in a nonpolar solvent at room temperature.

About 1 eq. to about 2 eq. of a Grignard reagent (XLIII) is added to a solution of 5-(substituted phenyl)-hexa-2,4-dien-1-al (XLII) in a polar aprotic solvent that is maintained at about −25° C. to about 10° C. The solution is stired for about 1 h to about 6 h to form a 6-(substituted phenyl)-hepta-3,5-dien-2-ol (XLIV).

The allylic alcohol of 6-(substituted phenyl)-hepta-3,5-dien-2-ol (XLIV) can be oxidized to a ketone by treating it with NMO and TRAP as described above to form an optionally substituted 6-(substituted phenyl)-hepta-3,5-dien-2-one (XXII).

The 6-(substituted phenyl)-hepta-3,5-dien-2-one (XXII) can be treated as in Scheme V, step 2 to form an optionally substituted 3-methyl-7-(substituted phenyl)-octa-2,4,6-trienoic acid alkyl ester (XXIV). The 3-methyl-7-(substituted phenyl)-octa-2,4,6-trienoic acid alkyl ester (XXIV) can be treated with an alkali hydroxide as in Scheme III, step 2 to form an optionally substituted 3-methyl-7-(substituted phenyl)-octa-2,4,6-trienoic acid (XX).

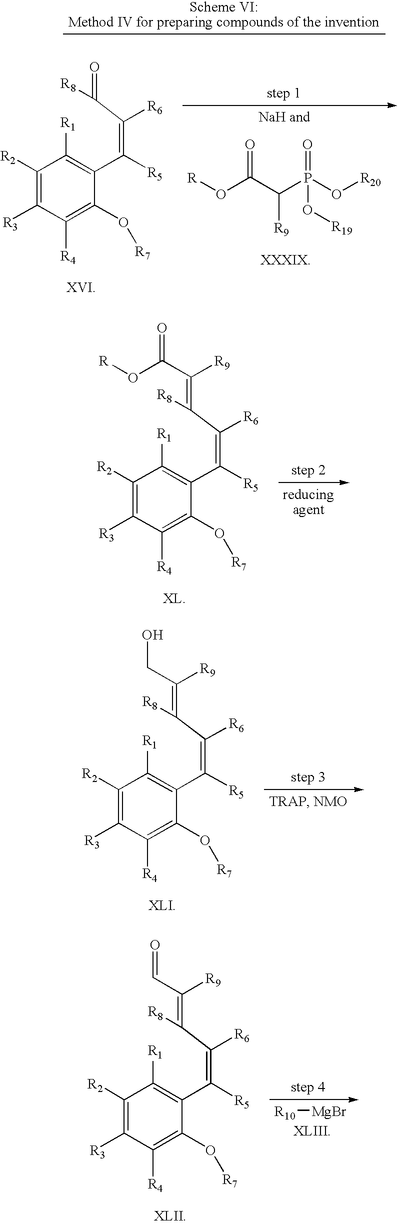

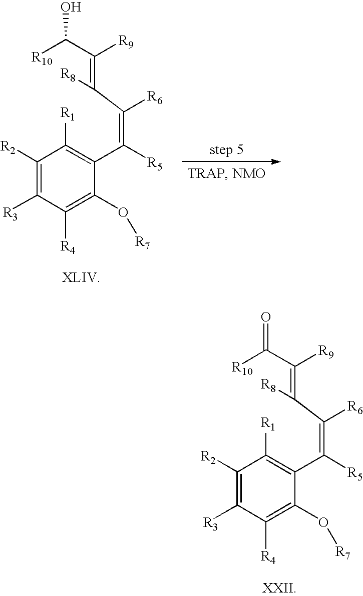

Compounds of the invention can also be prepared from an optionally substituted 2-acetylphenol (XXVII) (see Schemes VIII and IX). The 2-acetylphenol (XXVII) is prepared by cooling a solution of 2-halophenol (XXV) in an aprotic solvent to about −50° C. to about −100° C. then adding about 2.5 eq. of an alkyl lithium compound, such as n-butyl lithium, iso-butyl lithium or tert-butyl lithium. After about 15 min. to about 1 h, the solution is warmed to room temperature and stirred for about 1 h to about 4 h. The solution is then cooled to about −50° C. to about −100° C., and an excess of an alkyl acetate (XXVI) that is optionally substituted with from one to three fluoro groups is added. The solution is then allowed to warm to about −20° C. to about 10° C. and stirred for about 15 min. to about 2 h to afford the optionally substituted 2-acetylphenol (XXVII) (see Scheme VII).

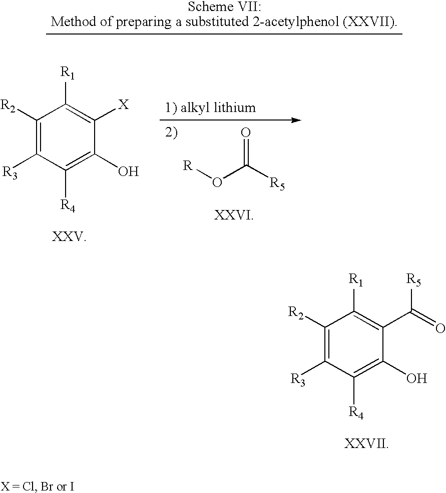

3-Methyl-7-(substituted phenyl)-octa-2,4,6-trienes in which R₅ and R₆ are in a cis configuration can be prepared from an optionally substituted 2-acetylphenol (XXVII) using the method depicted in Scheme VIII. In this method, a solution of trialkyl phosphonoacetate (XXVIII) in an aprotic solvent at about −25° C. to about 10° C. is treated with about 1 eq. to about 1.5 eq. of sodium hydride. After about 0.5 h to about 1.5 h, the optionally substituted 2-acetylphenol (XXVII) is added and the mixture is stirred for about 4 h to about 24 h to form a substituted coumarin (XXIX).

The substituted coumarin (XXIX) is treated with a reducing agent, such as sodium borohydride, lithium aluminum hydride or diisobutylaluminum hydride, to form a substituted 2-(4-hydroxybut-2-en-2-yl) phenol (XXX). The reaction is typically carried out in a polar solvent at about −25° C. to about 10° C. About 1 eq. to about 5 eq. of the reducing agent is used with respect to the coumarin (XXIX). Typically, the reaction is followed by thin layer chromatography (TLC) to determine when the reaction is complete.

The phenol hydroxy group is alkylated to form an optionally substituted 3-(substituted phenyl)-but-2-en-1-ol (XXXII) by treating the substituted 2-(4-hydroxybut-2-en-2-yl) phenol (XXX) in the presense of cesium fluoride or cesium carbonate with an optionally substituted alkyl halide or an optionally substituted alkenyl halide (R₇-X which represents the alkyl halide or alkenyl halide is referred to herein as “an aliphatic halide”) (XXXI). The reaction is carried out in a polar solvent at ambient temperatures. The aliphatic halide (XXXI) is present in about 1.1 eq. to about 2 eq. with respect to the 2-(4-hydroxybut-2-en-2-yl) phenol (XXX) and the cesium fluoride or cesium carbonate is present in about 1.5 eq. to about 3 eq. Typically, the reaction is followed by TLC to determine when the reaction is complete.

The allylic hydroxy group of 3-(substituted phenyl)-but-2-en-1-l (XXXII) is converted to an aldehyde to form an optionally substituted 3-(substituted phenyl)-but-2-en-1-al (XXXIII) by treatment with about 1 eq. to about 2 eq. of NMO and a cataylic amount of TPAP (about 0.01 eq. to about 0.1 eq.). The reaction is carried out in a nonpolar solvent at room temperature.

An anion of a trialkyl 3-methylphosphocrotonate (XXXIV) is formed by treating the trialkyl 3-methylphosphocrotonate (XXXIV) in a solution of a polar aprotic solvent maintained at about −50° C. to about −100° C. with about 1 eq. to about 1.5 eq. of an alkyl lithium. After addition of the alkyl lithium, the mixture is stirred for about 10 min. to about 30 min., then 3-(substituted phenyl)-but-2-en-1-al (XXXIII) is added to the mixture. The solution is allowed to warm up to room temperature to form an optionally substituted 3-methyl-7-(substituted phenyl)-octa-2,4,6-trienoic acid alkyl ester (XXXV) in which R₅ and R₆ are in a cis configuration. The 3-methyl-7-(substituted phenyl)-octa-2,4,6-trienoic acid alkyl ester (XXXV) can be treated with an alkali hydroxide as in Scheme III, step 2 to form an optionally substituted 3-methyl-7-(substituted phenyl)-octa-2,4,6-trienoic acid (XX).

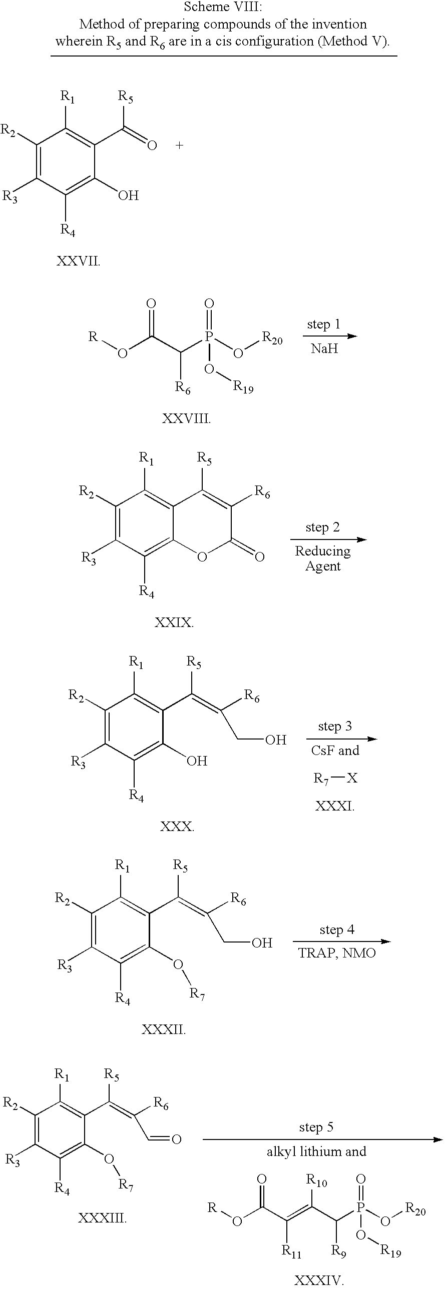

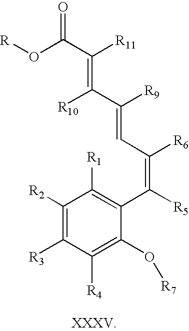

To prepare compounds of the invention in which R₅ and R₆ are in the trans configuration (see Scheme IX), an optionally substituted 2-acetylphenol (XXVII) in a polar aprotic solvent maintained at about −25° C. to about 10° C. is treated with about 1 eq. to about 1.5 eq. of sodium hydride to form an anion. About 1 eq. to about 2 eq. of an optionally substituted alkyl halide or alkenyl halide (XXXI) is added to the mixture. The reaction is allowed to warm up to room temperature and stirred for about 24 h to about 72 h more to form an optionally substituted 2-acetylphenyl aliphatic ether (XXXVI).

An anion of a trialkyl phosphonoacetate (XXVIII) is formed by treating a trialkyl phosphonoacetate (XXXVI) in a solution of an aprotic solvent maintained at about −25° C. to about 10° C. with about 1 eq. to about 1.5 eq. of sodium hydride. After about 0.5 h to about 1.5 h, the optionally substituted 2-acetylphenol (XXVII) is added, and the mixture is allowed to warm to room temperature and stirred for about 8 h to about 24 h to form an optionally substituted 3-(substituted phenyl)-but-2-enoic acid alkyl ester (XXXVII) as a mixture of isomers in which the major product is an isomer wherein R₅ and R₆ are in the trans configuration.

The 3-(substituted phenyl)-but-2-enoic acid alkyl ester (XXXVII) is treated with a reducing agent, such as sodium borohydride, lithium aluminum hydride or diisobutylaluminum hydride, to form an optionally substituted 3-(substituted phenyl)-but-2-en-1-ol (XXXVIII). The reaction is typically carried out in a polar solvent at about −25° C. to about 10° C. About 1 eq. to about 5 eq. of the reducing agent is used with respect to the 3-substituted phenyl)-but-2-enoic acid alkyl ester (XXXVII). Typically, the reaction is followed by thin layer chromatography (TLC) to determine when the reaction is complete.

The 3-(substituted phenyl)-but-2-en-1-ol (XXXVIII) can be treated as in Scheme VIII, steps 4 and 5 to form an optionally substituted 3-methyl-7-(substituted phenyl)-octa-2,4,6-trienoic acid alkyl ester (XXXV) in which R₅ and R₆ are in a trans configuration. The 3-methyl-7-(substituted phenyl)-octa-2,4,6-trienoic acid alkyl ester (XXXV) can be treated with an alkali hydroxide as in Scheme III, step 2 to form an optionally substituted 3-methyl-7-(substituted phenyl)-octa-2,4,6-trienoic acid (XX).

Example 5 was prepared by the method depicted in Scheme IX.

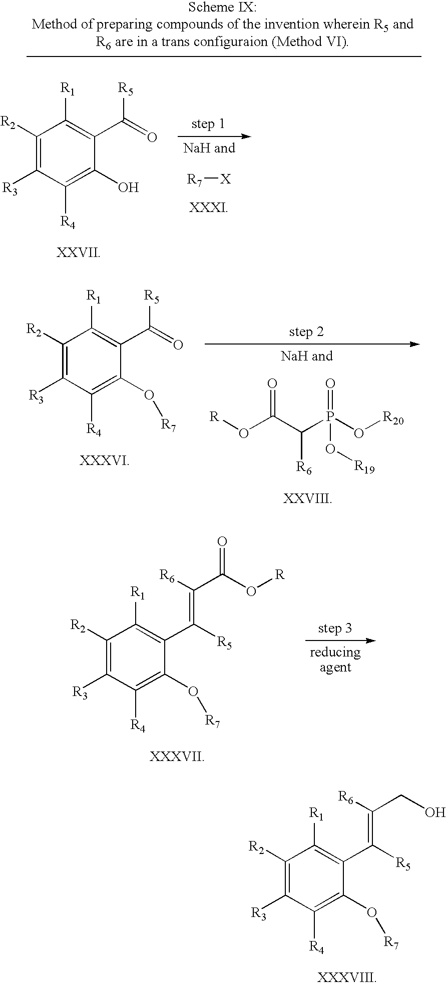

Methods of converting a 3-methyl-7-(substituted phenyl)-octa-2,4,6-trienoic acid or a 3-methyl-7-(substituted phenyl)-octa-2,4,6-trienoic acid alkyl ester to an anhydride are known to those skilled in the art. For example, a 3-methyl-7-(substituted phenyl)-octa-2,4,6-trienoic acid can be converted to an anhydride via an exchange reaction with an ester (see March, Advanced Organic Chemistry, 3^{rd} Edition (1985), John Wiley & Sons, pages 355-356, the entire teachings of which are encorporated herein by reference).

Methods of converting a 3-methyl-7-(substituted phenyl)-octa-2,4,6-trienoic acid alkyl ester to an amide are also known to those skilled in the art. For example, a 3-methyl-7-(substituted phenyl)-octa-2,4,6-trienoic acid alkyl ester can be converted to an amide by reacting it with ammonia or a primary or secondary amine (see March, Advanced Organic Chemistry, 3^{rd} Edition (1985), John Wiley & Sons, page 375, the entire teachings of which are encorporated herein by reference).

### EXAMPLES

General Procedures:

All reagents were obtained from commercial suppliers and used without further purification. Solvents were obtained anhydrous from commercial suppliers and used without further purification. ¹H spectra were recorded on a Varian 500 while or a Bruker Avance 250 as noted. Chemical shifts are reported in ppm (δ) and coupling constants (J) are reported in Hertz. Mass Spectra was obtained on a Micromass ZMD, and combustion analysis on an Exeter CE-440.

### Example 1

### 7-[3,5-di-tert-butyl-2-(2,2-difluoroethoxy)-phenyl]4-fluoro-3-methyl-octa-2,4,6-trienoic acid

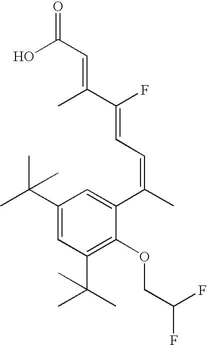

A. 1,5-Di-tert-butyl-2-(2,2-difluoroethoxy)-3-4-phenylsulfonyl-4-fluoro-1-methyl-buta-1,3-dienyl)-benzene

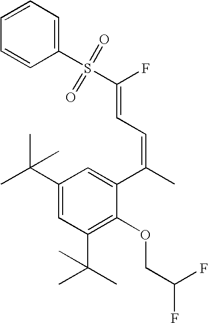

Fluoromethyl phenyl sulfone (1.03 g, 5.9 mmol) was dissolved in tetrahydrofuran (THF) (10 ml) and cooled to −78° C. under a nitrogen atmosphere. To this mixture was added diethyl chlorophosphate (0.854 ml, 5.9 mmol) followed by lithium hexamethyldisilazane (11.8 ml, 1.0 M soln., 11.8 mmol). This solution was stirred for 30 min., then a solution of 3-[3,5-di-tert-butyl-2-(2,2-difluoroethoxy)-phenyl]-but-2-enal (1.0 g, 2.95 mmol) in 10 ml of THF was added. The solution was left to warm to ambient temperature overnight, then the reaction was quenched with saturated ammonium chloride solution and extracted with ethyl acetate (2×30 ml). The combined organics were dried over MgSO₄, filtered and concentrated to yield 1,5-di-tert-butyl-2-(2,2-difluoroethoxy)-3-(4-phenylsulfonyl-4-fluoro-1-methyl-buta-1,3-dienyl)-benzene as a yellow solid, which was used without further purification.

B. Tributyl-{4-[3,5-di-tert-butyl-2-(2,2difluoroethoxy)-phenyl]-1-fluoro-penta-1,3-dienyl}-stannane

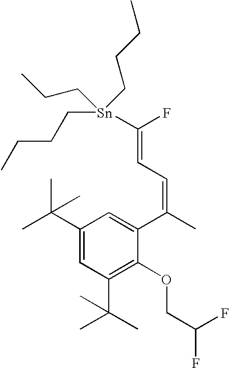

Tributyl tin hydride (1.75 ml, 6.49 mmol) and 2,2′-azobisisobutyronitrile (AIBN) (10 mg) were added to a solution of 1,5-di-tert-butyl-2-(2,2-difluoroethoxy)-3-(4-phenylsulfonyl4-fluoro-1-methyl-buta-1,3dienyl)-benzene (1.46 g, 2.95 mmol) in benzene. This mixture was heated to reflux for 10 hrs., then the reaction was concentrated to a residue. The residue purified by silica gel chromatography (0.1% ethyl acetate in hexanes) to give tributyl-{4-[3,5-di-tert-butyl-2-(2,2-difluoroethoxy)-phenyl]-1-fluoro-penta-1,3-dienyl}-stannane as a clear oil (108.9 mg, 6%).

¹H NMR (500 MHz, CDCl₃): δ 7.28 (d, 1H, J=2.5), 6.94 (d, 1H, J=2.5),6.57 (d, 1H, J=11.1), 5.99 (tt, 1H, J=4.1, J=57.5), 5.43, (dd, 1H, J=11.1, J=52.4), 4.10 (m, 1H), 3.87 (m, 1H), 2.16 (s, 3H), 1.45 (m, 6H), 1.40 (s, 9H), 1.30 (s, 9H), 1.25 (m, 6H), 0.92 (m, 6H), 0.83 (m, 9H).

B. 7-[3,5-Di-tert-butyl-2-(2,2-difluoroethoxy)-phenyl]4-fluoro-3-methyl-octa-2,4,6-trienoic acid

Tributyl-{4-[3,5-di-tert-butyl-2-(2,2-difluoroethoxy)-phenyl]-1-fluoro-penta-1,3-dienyl}-stannane (108 mg, 0.17 mmol) was dissolved in N,N-dimethyl formamide (DMF) (5 ml) along with 3-iodo-but-2-enoic acid (43 mg, 0.20 mmol) [prepared via literature procedure: Le Noble, W. J. JACS, 83, 1961, pp.3897-3899]. Nitrogen was bubbled into this mixture for 30 min., then dichlorobis(triphenylphosphine)-palladium(II) (11.8 mg, 0.017 mmol) was added, and the mixture heated to 80° C. under nitrogen for 2 hrs. The reaction was cooled, then poured into a solution of 620 mg of potassium fluoride in 5 mL of water. After the solution had stirred for 1 hr., the mixture was filtered, then extracted with ether (2×10 mL). The combined organic layers were dried over MgSO₄, filtered and concentrated to a residue. The residue was then purified by silica gel chromatography to give 7-[3,5-di-tert-butyl-2-(2,2-difluoroethoxy)-phenyl]-4-fluoro-3-methyl-octa-2,4,6-trienoic acid as a yellow solid (59.6 mg, 81%).

¹H NMR (250 MHz, CDCl₃): δ 7.33 (d, 1H, J=2.4), 6.98 (d, 1H, J=2.4), 6.59 (d, 1H, J=11.4), 6.29 (s, 1H), 5.99 (tt, 1H, J=4.1, J=57.5), 5.82, (dd, 1H, J=11.4, J=34.6), 4.10 (m, 1H), 3.87 (m, 1H), 2.26 (s, 3H), 2.07 (s, 3H), 1.43 (s, 9), 1.32 (s, 9H). MS [EI−] 437 (M−H)⁻.

### Example 2

### 7-[3,5-Di-tert-butyl-2-(2,2-difluoroethoxy)-phenyl]-5-fluoro-3-methyl-octa-2,4,6-trienoic acid

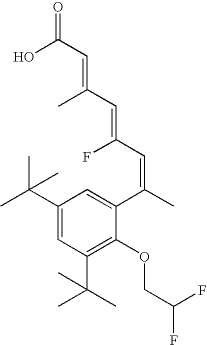

A. [3,5-Di-tert-butyl-2-(2,2-difluoroethoxy)-phenylethynyl]-trimethyl-silane

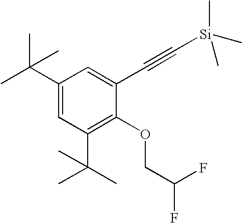

Dichlorobis(triphenylphosphine)palladium(II) (780 mg, 1.11 mmol), copper(I) iodide (211 mg, 1.11 mmol), and triethyl amine (6.19 ml, 44.4 mmol) were added to a solution of 1,5-di-tert-butyl-2-(2,2-difluoroethoxy)-3-iodo-benzene (4.40 g, 11.1 mmol) in dioxane (50ml) under an atmosphere of nitrogen. After stirring for 10 min., trimethylsilyl acetylene (3.14 ml, 22.2 mmol) was added, and the reaction was heated to 80° C. in a sealed tube. After 10 hrs., the reaction was cooled, poured into brine (50 mL), then extracted with ethyl acetate (2×30 mL). The organic layers were dried over MgSO₄, filtered, then concentrated to a residue. The residue was then purified by silica gel chromatography (1% ether in hexanes) to give [3,5-di-tert-butyl-2-(2,2-difluoro-ethoxy)-phenylethynyl]-trimethyl-silane as a yellow oil (1.40 g, 34%).

¹H NMR (250 MHz, CDCl₃): δ 7.12 (m, 2H, 6.03 (tt, 1H, J=4.1, J=57.5), 4.30 (td, 2H, J=4.1, J=13.1), 1.18 (s, 9H), 1.10 (s, 9H), 0.09 (s, 3H).

B. {2-[3,5-Di-tert-butyl-2-(2,2-difluoroethoxy)-phenyl]-propenyl}-trimethyl-silane

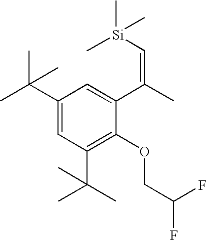

Dimethyl zinc (15.28 ml, 15.3 mmol) was added dropwise to a mixture of [3,5-di-tert-butyl-2-(2,2 difluoroethoxy)-phenylethynyl]-trimethyl-silane (1.4 g, 3.82 mmol) and nickel(II) acetylacetonate (245 mg, 0.95 mmol) in THF (60 ml) and 1-methyl-2-pyrrolidinone (NMP) (20 ml) that had been cooled to 0° C. under a nitrogen atmosphere. After complete addition, the reaction was allowed to warm to ambient temperature overnight. The reaction was poured into an ice/sat. ammonium chloride mixture and stirred for 10 min., then filtered and extracted with ethyl acetate (3×50 mL). The combined organic layers were combined, dried over MgSO₄, filtered, then concentrated to a residue. The residue was purified by silica gel chromatography (0.1% ethyl acetate in hexanes) to give {2-[3,5-di-tert-butyl-2-(2,2-difluoroethoxy)-phenyl]-propenyl}-trimethyl-silane as a clear oil (95.6 mg, 67%).

¹H NMR (250 MHz, CDCl₃): δ 7.43 (s, 1H), 7.08 (d, 1H, J=2.5), 6.22 (tt, 1H, J=4.2, J=55.4), 5.84 (d, 1H, J=1.3), 4.50 (m, 1H), 4.15 (m, 1H), 2.38 (d, 3H, 1.3), 1.56 (s, 9H), 1.46 (s, 9H), 0.00 (s, 3H).

C. 1,5-Di-tert-butyl-2-(2,2-difluoroethoxy)-3-(2-iodo-1-methylvinyl)-benzene

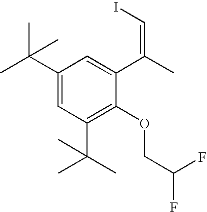

Iodine monochloride (40.6 mg, 0.28 mmol) was added to a solution of {2-[3,5-di-tert-butyl-2-(2,2-difluoroethoxy)-phenyl]-propenyl}-trimethyl-silane (95.6 mg, 0.25 mmol) in carbon tetrachloride (5 ml) that had been cooled to 0° C. under a nitrogen atmosphere. After 2 hrs., the reaction was poured into a 10% sodium sulfate solution (5 mL) and extracted with dichloromethane (2×10 mL). The combined organic layers were dried over MgSO₄, filtered, and concentrated to a residue. The residue was purified by silica gel chromatography (1% ethyl acetate in hexanes) to give 1,5-di-tert-butyl-2-(2,2-difluoroethoxy)-3-(2-iodo-1-methylvinyl)-benzene as a clear oil (23.9 mg, 22%).

¹NMR (250 MHz, CDCl₃): δ 7.25 (d, 1H, J=2.5), 6.93 (d, 1H, J=2.5), 6.08 (d, 1H, J=1.5),5.97 (tt, 1H, J=4.1, J=55.2), 3.99 (m, 2H), 2.02 (d, 3H, 1.3), 1.33 (s, 9H), 1.24 (s, 9H).

D. 5-Benzenesulfonyl-5-fluoro-3-methyl-penta-2,4-dienoic acid ethyl ester

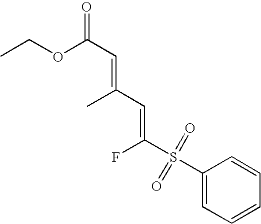

Diethyl chlorophosphate (4.24 ml, 29.4 mmol) followed by lithium hexamethyldisilazane (58.75 ml, 1M soln., 58.8 mmol) was added to a solution of fluoromethyl phenyl sulfone (5.12 g, 29.4 mmol) in THF (30 ml) that had been cooled to −78° C. under a nitrogen atmosphere. After 30 min., a solution of ethyl 3-methyl4-oxocrotonate (2.0 ml, 14.7 mmol) in 10 mL of THF was added, and the reaction was allowed to warm to ambient temperature overnight. The reaction was quenched with saturated ammonium chloride solution and extracted with ethyl acetate (2×50 mL). The combined organic layers were dried over MgSO₄, filtered and concentrated to yield 5-benzenesulfonyl-5-fluoro-3-methyl-penta-2,4-dienoic acid ethyl ester as a brown solid which was used without further purification.

E. 5-Fluoro-3-methyl-5-tributylstannanyl-penta-2,4-dienoic acid ethyl ester

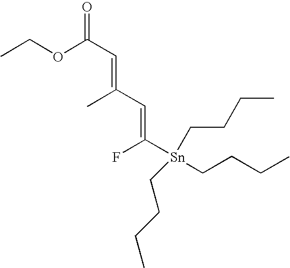

Tributyl tin hydride (8.69 ml, 32.3 mmol) and AIBN (10 mg) were added to a solution of 5-benzenesulfonyl-5-fluoro-3-methyl-penta-2,4-dienoic acid ethyl ester (4.38 g, 14.7 mmol) in benzene (50 mL). This mixture was heated to reflux for 10 hrs., then the reaction was concentrated to a residue. The residue was purified by silica gel chromatography (1% ethyl acetate in hexanes) to give 5-fluoro-3-methyl-5-tributylstannanyl-penta-2,4-dienoic acid ethyl ester as a clear oil (57.9 mg, 1%).

¹H NMR (250 MHz, CDCl₃): δ 6.98 (d, 1H, J=61.4), 5.48 (s, 1H), 4.17 (q, 2H, J=6.8), 2.20 (d, 3H, J=1.2), 1.59 (m, 6H), 1.37 (m, 6H), 1.30 (t, 3H, J=6.8), 1.12 (m, 6H), 0.92 (t, 9H, J=7.5).

F. 7-[3,5-Di-tert-butyl-2-2,2-difluoroethoxy)-phenyl]-5-fluoro-3-methyl-octa-2,4,6-trienoic acid ethyl ester

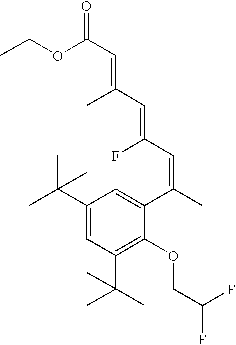

Nitrogen was bubbled through a mixture of 1,5-di-tert-butyl-2-(2,2-difluoro-ethoxy)-3-(2-iodo-1-methylvinyl)-benzene (24 mg, 0.06 mmol) and 5-fluoro-3-methyl-5-tributylstannanyl-penta-2,4-dienoic acid ethyl ester (30 mg, 0.07 mmol) in DMF (5 ml). Dichlorobis(triphenylphosphine)palladium(II) (4 mg, 0.006 mmol) was added to the mixture and it was heated to 80° C. under nitrogen. After 2 hrs., the reaction was cooled, then poured into a solution of 620 mg of potassium fluoride in 5 mL of water. After the mixture had stirred for 1 hr., it was filtered, then extracted with ether (2×10 mL). The organic layers were combined, dried over MgSO₄, filtered, and concentrated to a residue. The residue was purified by silica gel chromatography (1% ethyl acetate in hexanes) to give 7-[3,5-di-tert-butyl-2-(2,2-difluoroethoxy)-phenyl]-5-fluoro-3-methyl-octa-2,4,6-trienoic acid ethyl ester as a clear oil. This material was used without further purification.

G. 7-.[3,5-Di-tert-butyl-2-(2,2-difluoroethoxy)-phenyl]-5-fluoro-3-methyl-octa-2,4,6-trienoic acid

A solution of 7-[3,5-di-tert-butyl-2-(2,2-difluoroethoxy)-phenyl]-5-fluoro-3-methyl-octa-2,4,6-trienoic acid ethyl ester in methanol (5 ml) and 1N NaOH (5 ml) was heated to 60° C. After 4 hrs., the reaction was cooled and brought to pH 3, then extracted with ethyl acetate (2×10 mL). The combined organic layers were then dried over MgSO₄, filtered and concentrated to a residue. The residue purified by silica gel chromatography (10% ethyl acetate in hexanes) to give 7-[3,5-di-tert-butyl-2-(2,2-difluoroethoxy)-phenyl]-5-fluoro-3-methyl-octa-2,4,6-trienoic acid as a white solid (7.2 mg, 36%).

¹H NMR (250 MHz, CDCl₃): δ 7.87 (d, 1H, J=2.4), 7.63 (d, 1H, J=2.4), 6.92 (d, 1H, J=1.3), 6.53 (dd, 1H, J=1.3, J=11.9), 6.01 (tt, 1H, J=4.1, J=57.5), 5.92, (d, 1H, J=30.9), 4.00 (m, 1H), 3.97 (m, 1H), 2.29 (s, 3H), 2.07 (s, 3H), 1.39 (s, 9H), 1.36 (s, 9H). MS [EI−] 437 (M−H)³¹ .

### Example 3

### (2Z,4E,6Z)-7-(2-Butoxy-3,5-diisopropylphenyl)-3-trifluoromethyl-octa-2,4,6-trienoic acid

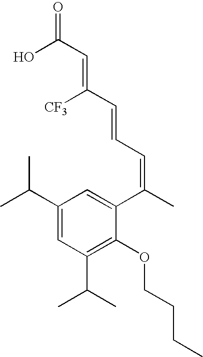

A. 6-(2-Butoxy-3,5-diisopropylphenyl)-1,1,1-trifluoro-hepta-3,5-dien-2-one

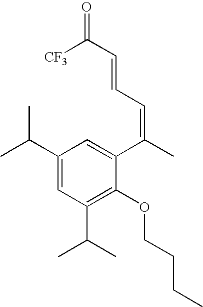

Piperidine (40 mg, 0.47mmol) followed by glacial acetic acid (40 mg, 0.67mmol) was added to a solution of 3-(2-butoxy-3,5-diisopropyl-phenyl)-but-2-enal (168 mg, 0.556 mmol) in THF (6 ml). Then trifluoromethyl acetone (2 mL) was added in one portion. The reaction was stirred for 1 hr. at room temperature, then quenched with saturated ammonium chloride solution and concentrated in vacuo to a residue. The residue was partitioned between ethyl acetate and water. The organic layer was washed with saturated ammonium chloride solution and brine, then dried over sodium sulfate, filtered and concentrated in vacuo to a residue. The residue was then purified by silica gel chromatography (30-100% toluene in hexanes) to give 6-(2-butoxy-3,5-diisopropyl-phenyl)-1,1,1-trifluoro-hepta-3,5-dien-2-one (70 mg, 32%).

¹H NMR (400 MHz, CDCl₃) δ 7.45 (dd, 1H, J=15,17), 7.0 (d, 1H, J=1), 6.6 (d, 1H, J=1), 6.3 (d, 2H, J=15), 3.5 (t, 2H, J=9), 3.2 (m, 1H), 2.75 (m, 1H), 2.2 (s, 3H), 1.55 (m, 2H), 1.35 (m, 2H), 1.15 (d, 12H), 0.8 (t, 3H, J=8).

B. (2Z,4E,6Z)-7-(2-Butoxy-3,5-diisopropylphenyl)-3-trifluoromethyl-octa-2,4,6-trienoic acid methyl ester and (2E,4E,6Z)-7-(2-Butoxy-3,5-diisopropylphenyl)-3-trifluoromethyl-octa-2,4,6-trienoic acid methyl ester

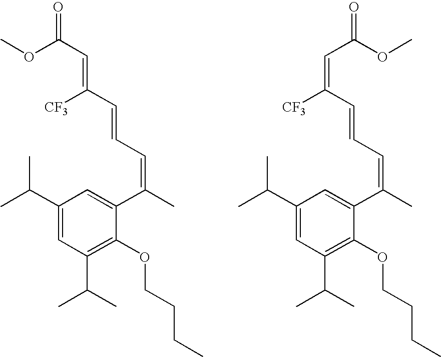

NaH (40 mg, 1.11 mmol) was added to a solution of trimethyl phosphonoacetate (0.18 mL, 1.11 mmol) in diethyl ether (10 ml). After stirring at room temperature for 1 hour, a solution of 6-(2-butoxy-3,5-diisopropyl-phenyl)-1,1,1-trifluoro-hepta-3,5dien-2-one(200 mg, 0.504 mmol) in diethyl ether (5 ml) was added, and the mixture was stirred at ambient temperature overnight. The reaction was quenched with water and concentrated in vacuo to a residue. The residue was dissolved in ethyl acetate and washed with water and brine. The organic layer was dried over sodium sulfate, filtered and concentrated in vacuo to a residue that was purified by silica gel chromatography (30-100% toluene in hexanes) to give (2Z,4E,6Z)-7-(2-butoxy-3,5-diisopropylphenyl)-3-trifluoromethyl-octa-2,4,6-trienoic acid methyl ester (30 mg, 13%) and (2E,4E,6Z)-7-(2-butoxy-3,5-diisopropylphenyl)-3-trifluoromethyl-octa-2,4,6-trienoic acid methyl ester (161 mg, 48%).

(2Z,4E,6Z)-7-(2-butoxy-3,5-diisopropylphenyl)-3-trifluoromethyl-octa-2,4,6-trienoic acid methyl ester:

¹H NMR (400 MHz, CDCl₃) δ 6.95 (d, 1H, J=1), 6.6 (d, 1H, J=1), 6.55 (dd, 1H, J=12, 15), 6.1 (d, 1H, J=12), 6.0 (s, 1H), 5.98 (d, 1H, J=15), 3.65 (s, 3H), 3.5 (t, 2H, J=9),3.2 (m, 1H), 2.75 (m, 1H), 2.1 (s, 3H), 1.55 (m, 2H), 1.35 (m, 2H), 1.15 (m, 12H), 0.8 (t, 3H, J=9).

(2E,4E,6Z)-7-(2-butoxy-3,5-diisopropylphenyl)-3-trifluoromethyl-octa-2,4,6-trienoic acid methyl ester:

¹H NMR (400 MHz, CDCl₃) δ 7.3 (d, 1H, J=17), 6.9 (d, 1H, J=2), 6.65 (dd, 1H, J=17,12), 6.6 (d, 1H, J=2), 6.2 (d, 1H, J=12), 6.0 (s, 1H), 3.7 (s, 3H), 3.5 (br t, 1H), 3.2 (m, 1H), 2.75 (m, 1M), 2.15 (s, 3H), 1.55 (m, 2H), 1.35 (m, 2H), 1.15 (m, 12H), 0.8 (t, 3H, J=9).

C. (2Z,4E,6Z)-7-(2-Butoxy-3,5-diisopropylphenyl)-3-trifluoromethyl-octa-2,4,6-trienoic acid

An aqueous solution of 1M LiOH (0.13 ml, 0.132 mmol) was added to a solution of (2Z,4E,6Z)-7-(2-butoxy-3,5-diisopropyl-phenyl)-3-trifluoromethyl-octa-2,4,6-trienoic acid methyl ester (30 mg, 0.066 mmol) in methanol (5 ml). The reaction was heated to 50° C. overnight, then concentrated in vacuo to a residue. The residue was dissolved in ethyl acetate and washed with 1N HCl and brine. The organic layer was dried over sodium sulfate, filtered, and concentrated in vacuo to a residue. The residue was purified by silica gel chromatography (25% ethyl acetate in toluene) to give (2Z,4E,6Z)-7-(2-butoxy-3,5-diisopropylphenyl)-3-trifluoromethyl-octa-2,4,6-trienoic acid (21 mg, 72%).

¹H NMR (400 MHz, CDCl₃) δ 6.95 (d, 1H, J=1), 6.6 (m, 2H), 6.15 (d, 1H, J=11), 6.0 (d, 2H, J=15), 3.5 (t,2H, J=8), 3.2 (m, 1H), 2.75 (m, 1H), 2.1 (s, 3H), 1.55 (m, 2H), 1.35 (m, 2H), 1.15 (m, 12H), 0.8 (t, 3H, J=9.5). MS [EI+]: 439 (m+H)⁺, [EI−]: 437 (m−H)⁻.

### Example 4

### (2E,4E,6Z)-7-(2-Butoxy-3,5-diisopropylphenyl)-3-trifluoromethyl-octa-2,4,6-trienoic acid

An aqueous solution of 1M LiOH (0.35 mL, 0.712 mmol) was added to a solution of (2E,4E,6Z)-7-(2-butoxy-3,5-diisopropylphenyl)-3-trifluoromethyl-octa-2,4,6-trienoic acid methyl ester (161 mg, 0.356 mmol) (prepared in Example 3, step B) in methanol (5 ml). The reaction was stirred at room temperature overnight, then heated to 50° C. for 1 hr. The reaction was then concentrated in vacuo to a residue. The residue was dissolved in ethyl acetate and washed with 1N HCl and brine. The organic layer was dried over sodium sulfate, filtered, and concentrated in vacuo to give (2E,4E,6Z)-7-(2-butoxy-3,5-diisopropylphenyl)-3-trifluoromethyl-octa-2,4,6-trienoic acid.

MS [EI+]: 439 (m+H)⁺, [EI−]: 437 (m−H)⁻. Combustion Analysis for C25H₃₃F₃O₃: Calculated: C, 68.4731; H. 7.5850. Found: C, 69.10,; H, 7.79.

### Example 5

### (2E,4E,6E)-3-methyl-7-(2-ethoxy-3,5-di-tert-butylphenyl)-8,8,8-trifluoroocta-2,4,6-trienoic acid

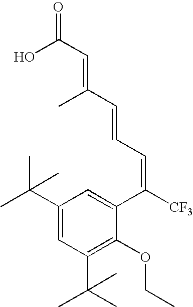

A. 2,2,2-Trifluoro-1-(2-hydroxy-3,5-di-tert-butylphenyl)-ethanone

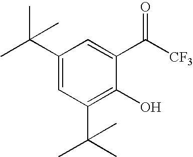

Into a flame-dried 200 mL round-bottomed flask fitted for magnetic stirring was added 2-bromo4,6-di-tert-butylphenol (5.0 g, 17.53 mmoles) and diethyl ether (88 mL). This solution was cooled to −78° C. and n-butyllithium (14.7 mL of a 2.5 M soln, 36.81 mmoles) was added dropwise via syringe. The reaction was subsequently stirred at −78° C. for 30 min and then gradually warmed to room temperature and stirred for 3 h. The solution was re-cooled to −78° C., and ethyl trifluoroacetate (6.26 mL, 52.59 mmoles) was added dropwise via syringe. This reaction was then slowly warmed to 0° C. and stirred for 30 min. At this time, the reaction was quenched with a saturated aqueous solution of ammonium chloride. This crude mixture was concentrated in-vacuo, extracted with hexanes, and filtered over a silica plug affording 4.15 g of 2,2,2-trifluoro-1-(2-hydroxy-3,5-di-tert-butylphenyl)-ethanone (13.73 mmoles, 78% yield).

¹H NMR (400 MHz, CDCl₃) δ: 11.60 (s, 1H), 7.72 (s, 1H), 7.63 (s, 1H), 1.44 (s, 9H), 1.32 (s, 9H).

B. 2,2,2-Trifluoro-1-(2-ethoxy-3,5-di-tert-butylphenyl)-ethanone

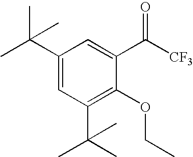

2,2,2-Trifluoro-1-(2-hydroxy-3,5-di-tert-butylphenyl)-ethanone (1.0 g, 3.31 mmoles) and DMF (33 mL) were added to a flame-dried 100 mL round-bottomed flask fitted for magnetic stirring. This solution was cooled to 0° C. and sodium hydride (0.132 g of a 60% suspension, 3.31 mmoles) was added. The reaction was subsequently stirred at 0° C. for 30 min. and then iodoethane (0.317 mL, 3.97 mmoles) was added dropwise via syringe. The reaction was then slowly warmed to room temperature and stirred for 72 h. At this time, the reaction was quenched with a saturated aqueous solution of ammonium chloride. The crude reaction mixture was extracted with hexanes and filtered over a silica plug affording 1.09 g of 2,2,2-trifluoro-1-(2-ethoxy-3,5-di-tert-butylphenyl)-ethanone (3.31 mmoles, quantitative yield).

¹H NMR (400 MHz, CDCl₃) δ: 7.62 (s, 1H), 7.44 (s, 1H), 3.79 (m, 2H), 1.40 (m, 12H), 1.32 (s, 9H).

C. 4,4,4-Trifluoro-3-(2-ethoxy, 3,5-di-tert-butylphenyl)-but-2-enoic acid methyl ester

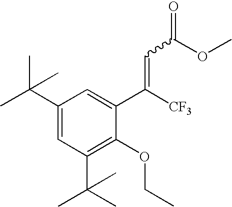

Trimethyl phosphonoacetate (1.34 mL, 8.28 mmoles) and DMF (33 mL) were added to a flame-dried 100 mL round-bottomed flask fitted for magnetic stirring. This solution was cooled 0° C. and sodium hydride (0.318 g of a 60% suspension, 7.94 mmoles) was added. The reaction was subsequently stirred at 0° C. for 30 min. 2,2,2-Trifluoro-1-(2-ethoxy-3,5-di-tert-butylphenyl)-ethanone (1.09 g, 3.31 mmoles) and DMF (5 mL) were then added dropwise via addition funnel. This reaction was slowly warmed to room temperature and stirred for 24 h. At this time, the reaction was quenched with a saturated aqueous solution of ammonium chloride. This crude mixture was extracted with hexanes and filtered over a silica plug affording 4,4,4-trifluoro-3-(2-ethoxy, 3,5-di-tert-butylphenyl)-but-2-enoic acid methyl ester. Analysis of this material by NMR indicated a mixture of isomers with one being the major product. The isomers were not separated and assigned until the last step of the synthesis. Thus, the mixture of isomers was carried on to the next step.

D. 4,4,4-Trifluoro-3-(2-ethoxy-3,5-di-tert-butylphenyl)-but-2-en-1-ol

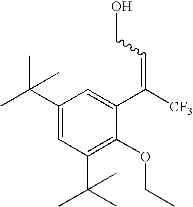

4,4,4-Trifluoro-3-(2-ethoxy, 3,5-di-tert-butylphenyl)-but-2-enoic acid methyl ester (crude, 3.31 max) and diethyl ether (30 mL) were added to a flame-dried 100 mL round-bottomed flask fitted for magnetic stirring. This solution was cooled to 0° C. and diisobutylaluminum hydride (hereinafter “DIBAL-H”) (4.41 mL of a 1.5M soln, 6.62 mmoles) was added dropwise via syringe. After the addition was complete, the reaction was quenched with a saturated aqueous solution of ammonium chloride. This crude mixture was extracted with hexanes and filtered over a silica plug affording crude 4,4,4-trifluoro-3-(2-ethoxy-3,5-di-tert-butylphenyl)-but-2-en-1-ol which was used without further purification.

E. 4,4,4-Trifluoro-3-(2-ethoxy-3,5-di-tert-butylphenyl)-but-2-enal

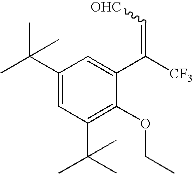

4,4,4-Trifluoro-3-(2-ethoxy-3,5-di-tert-butylphenyl)-but-2-en-1-ol (crude, 3.31 max), 4-methylmorpholine N-oxide (1.0 g, 8.53 mmoles) and CH₂Cl₂ (15 mL) were added to a flame-dried 30 mL round-bottomed flask fitted for magnetic stirring at room temperature. Tetrapropyl ammonium peruthenate (catalytic, spatula tip) was added to this solution, and the resultant black solution was stirred at room 20 temperature for 1 h. This solution was then passed directly over a short pad of silica and washed with dichloromethane affording crude 4,4,4-trifluoro-3-(2-ethoxy-3,5-di-tert-butylphenyl)-but-2-enal which was used without further purification.

F. (2E,4E,6E)-3-methyl-7-(2-ethoxy-3,5-di-tert-butylphenyl)-8,8,8-trifluoroocta-2,4,6-trienoic acid ethyl ester

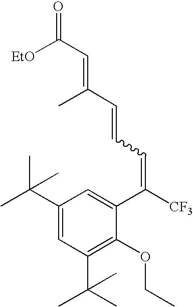

Triethyl-3-methyl-4-phosphonocrotonate (2.41 mL, 9.93 mmoles), THF (25 mL), and DMPU (5 mL) were,added to a flame dried round-bottomed flask. This solution was cooled to −78° C. and n-BuLi (3.84 mL of a 2.5M solution in hexanes, 9.60 mmoles) was added dropwise via syringe. The reaction was then allowed to stir for 30 min. at −78° C. At this time, 4,4,4-trifluoro-3-(2-ethoxy-3,5-di-tert-butylphenyl)-but-2-enal (3.31 mmoles max) was added in THF (10 mL), and the solution was allowed to stir at −78° C. for 2 h. Subsequently, the reaction was quenched with distilled water and extracted with a 10% ethyl acetate/hexanes solution. The organic layer was directly passed over a silica gel plug, and the ester was eluted using 10% ethyl acetate/hexanes. The filtrate was concentrated and dried in-vacuo affording crude (2E,4E,6E)-3-methyl-7-(2-ethoxy-3,5-di-tert-butylphenyl)-8,8,8-trifluoroocta-2,4,6-trienoic acid ethyl ester which was carried on to the final step without further purification.

G. (2E,4E,6E)-3-methyl-7-(2-ethoxy-3,5-di-tert-butylphenyl)-8,8,8-trifluoroocta-2,4,6-trienoic acid

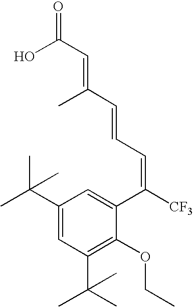

(2E,4E,6E)-3-Methyl-7-(2-ethoxy-3,5-di-tert-butylphenyl)-8,8,8-trifluoroocta-2,4,6-trienoic acid ethyl ester (3.31 mmoles max), ethanol (30 mL) and LiOH (4.97 mL of a 2N solution, 9.93 mmoles) was added to a 100 mL round-bottomed flask fitted with a reflux condenser. This solution was heated to reflux for 2 h. The resultant mixture was quenched with HCl(aq) and extracted twice with ethyl acetate. The organic layer was washed with brine, collected and filtered over a pad of Celite. The solvent was removed in-vacuo and the crude (2E,4E,6E)-3-methyl-7-(2-ethoxy-3,5-di-tert-butylphenyl)-8,8,8-trifluoroocta-2,4,6-trienoic acid was purified by reverse-phase preparative HPLC affording 9.0 mg (0.021 mmoles, 0.62% yield over 5-steps) of the desired isomer (as shown above) which was >99% pure by HPLC and NMR.

¹H NMR (400 MHz, CDCl₃) δ: 7.35 (s, 1H), 7.05 (s, 1H), 6.86 (d, J=10.8 Hz, 1H), 6.57 (d, J=15.6 Hz, 1H), 6.11 (d of d, J=15.3 Hz, J=10.9 Hz, 1H), 5.37 (s, 1H), 3.73 (m, 2H), 3.13 (s, 3H), 1.40 (s, 9H), 1.28 (s, 9H), 1.21 (m, 3H).

Biological Activity

### Example 6

### Evaluation of Retinoid Receptor Subfamily Activity In Vitro

Utilizing the “cis-trans” or “co-transfection” assay described by Evans et al., Science, 240:889-95 (May 13, 1988), the disclosure of which is herein incorporated by reference, the dimer-selective RXR modulator compounds of the present invention were tested and found to have strong, specific activity as selective RXR modulators, including activity as full agonists, partial agonists and/or full antagonists of RXR homodimers and/or heterodimers. This assay is described in further detail in U.S. Pat. Nos. 4,981,784 and 5,071,773, the disclosures of which are incorporated herein by reference.

The co-transfection assay provides a method for identifying functional agonists which mimic, or antagonists which inhibit, the effect of native hormones, and quantifying their activity for responsive IR proteins. In this regard, the co-transfection assay mimics an in vivo system in the laboratory. Importantly, activity in the co-transfection assay correlates very well with known in vivo activity, such that the co-transfection assay functions as a qualitative and quantitative predictor of a tested compounds in vivo pharmacology. See, e.g., T. Berger et al. 41 J. Steroid Biochem. Molec. Biol. 773 (1992), the disclosure of which is herein incorporated by reference.

In the co-transfection assay, cloned cDNA for one or more IRs (e.g. human RARα, RXRα, or PPARγ), alone or in combination (i.e. for heterodimer assays) under the control of a constitutive promoter (e.g., the SV 40, RSV or CMV promoter) is introduced by transfection (a procedure to introduce exogenous genes into cells) into a background cell substantially devoid of endogenous IRs. These introduced gene(s) direct the recipient cells to make the IR protein(s) of interest. A further gene is also introduced (co-transfected) into the same cells in conjunction with the IR gene(s). This further gene, comprising the cDNA for a reporter protein, such as firefly luciferase (LUC), controlled by an appropriate hormone responsive promoter containing a hormone response element (HRE). This reporter plasmid functions as a reporter for the transcriptional-modulating activity of the target IR(s). Thus, the reporter acts as a surrogate for the products (mRNA then protein) normally expressed by a gene under control of the target receptor(s) and their native hormone(s).

The co-transfection assay can detect small molecule agonists or antagonists, including partial agonists and antagonist, of target IRs. Exposing the transfected cells to an agonist ligand compound increases reporter activity in the transfected cells. This activity can be conveniently measured, e.g., by increasing luciferase production and enzymatic activity, which reflects compound-dependent, IR-mediated increases in reporter transcription. To detect antagonists, the co-transfection assay is carried out in the presence of a constant concentration of an known agonist to the target IR (e.g., 4-[(3,5,5,8,8-Pentamethyl-5,6,7,8-tetrahydro-2-naphthyl)ethenyl]benzoic acid (LGD1069, Ligand Pharmaceuticals, Inc.) for RXRα) known to induce a defined reporter signal. Increasing concentrations of an antagonist will decrease the reporter signal (e.g., luciferase production). The co-transfection assay is therefore useful to detect both agonists and antagonists of specific IRs. Furthermore, it determines not only whether a compound interacts with a particular IR, but whether this interaction mimics (agonizes) or blocks (antagonizes) the effects of native or synthetic regulatory molecules on target gene expression, as well as the specificity and strength of this interaction.

The activity of the dimer-selective RXR retinoid modulator compounds of the present invention were evaluated utilizing the co-transfection assay according to the following illustrative Examples.

### Example 6A

### RXR and RAR Binding

In addition to the cotransfection data, the binding of selected compounds of the present invention to the RAR and RXR receptors was also investigated according to the methodology described in M. F., Boehm, et al., “Synthesis and Structure-Activity Relation-ships of Novel Retinoid X Receptor Selective Retinoids”, 37 J. Med. Chem., 2930 (1994); M. F. Boehm, et al., “Synthesis of High Specific Activity [³H]-9-cis Retinoic Acid and Its Application for Identifying Retinoids with Unusual Binding Properties”, 37 J. Med. Chem., 408 (1994), and E. A. Allegretto, et al., “Characterization and Comparison of Hormone-Binding and Transactivation Properties of Retinoic Acid and Retinoid X Receptors Expressed in Mammalian Cells and Yeast”, 268 J. Biol. Chem., 22625 (1993), the disclosures of which are herein incorporated by reference.

Non-specific binding was defined as that binding remaining in the presence of 500 nM of the appropriate unlabelled compound. At the end of the incubation period, bound ligand was separated from free. The amount of bound tritiated retinoid was determined by liquid scintillation counting of an aliquot (700 μL) of the supernatant fluid or the hydroxylapatite pellet.

After correcting for non-specific binding, IC₅₀ values were determined. The IC₅₀ value is defined as the concentration of competing ligand needed to reduce specific binding by 50%. The IC₅₀ value was determined graphically from a log-logit plot of the data. The Kᵢ values were determined by application of the Cheng-Prussof equation to the IC₅₀ values, the labeled ligand concentration and the K_{d} of the labeled ligand.

The binding activity of RXRα, RXRβ, RXRγ, RARα, RARβ, and RARγ of selected compounds of the present invention are shown in Table 1 below.

TABLE 1
Binding activity of RXRα, RXRβ, RXRγ, RARα, RARβ, and
RARγ of selected compounds of the present invention

| | RAR Binding (nM) | | | RXR Binding (nM) | | |
|---|---|---|---|---|---|---|
| Example | alpha | beta | gamma | alpha | beta | gamma |
| 5 | >10000 | 7254 | >10000 | 38 | 52 | 184 |
| 3 | >10000 | >10000 | >10000 | 1304 | 203 | 662 |
| 4 | >10000 | >10000 | >10000 | 9112 | 1080 | 865 |
| 1 | 1152 | 6632 | >10000 | 4.8 | 12 | 21 |
| 2 | >10000 | 2753 | >10000 | 8.2 | 15 | 29 |

As can be seen in Table 1, most of the dimer-selective RXR modulator compounds displayed high affinity binding to RXRα, RXRβ, RXRγ, and little binding affinity for RARα, RARβ, and RARγ.

### Example 6B

### RXR Homodimer Co-Transfection Assay

CV-1 cells (African green monkey kidney fibroblasts) were cultured in the presence of Dulbecco's Modified Eagle Medium (DMEM) supplemented with 10% charcoal resin-stripped fetal bovine serum then transferred to 96-well microtiter plates one day prior to transfection.

To determine agonist and antagonist activity of the modulator compounds of the present invention, the CV-1 cells were transiently transfected by calcium phosphate coprecipitation according to the procedure of Berger et al., 41 J. Steroid Biochem. Mol. Biol., 733 (1992) with the receptor expressing plasmid pRShRXRα, Mangelsdorf et al., 345 Nature, 224 (1990), the disclosures of which are herein incorporated by reference at a concentration of 10 ng/well. The receptor expression plasmid was cotransfected along with a reporter plasmid at 50 ng/well, the internal control plasmid pRS-β-Gal at 50 ng/well and filler DNA, pGEM, at 90 ng/well.

The reporter plasmid CRBPIITKLUC, which contains an RXRE (retinoid X receptor response element, as described in Mangelsdorf et al., 66 Cell, 555 (1991), the disclosure of which is herein incorporated by reference, was used in transfections for the RXR homodimer assay. This reporter plasmid contains the cDNA for firefly luciferase (LUC) under the control of a promoter containing the RXR response element. As noted above, pRS-β-Gal, coding for constitutive expression of E. coli β-galactosidase (β-Gal), was included as an internal control for evaluation of transfection efficiency and compound toxicity.

Six hours after transfection, media was removed and the cells were washed with phosphate-buffered saline (PBS). Media containing compounds of the present invention in concentrations ranging from 10⁻¹⁰ to 10⁻⁵ M were added to the cells. Similarly, the reference compounds all-trans retinoic acid (ATRA)(Sigma Chemical), LGD1069 (4-[(3,5,5,8,8-Pentamethyl-5,6,7,8-tetrahydro-2-naphthyl)ethenyl]benzoic acid: Ligand Pharmaceuticals, Inc.) and LG100268 (6-[1-(3,5,5,8,8-pentamethyl-5,6,7,8-tetrahydronaphthalen-2-yl)cyclopropyl]nicotinic acid: Ligand Pharmaceuticals, Inc.), compounds with known agonist activity on RXRs, were added at similar concentrations to provide a reference point for analysis of the agonist activity of the compounds of the present invention. When determining the antagonist activity of the compounds of the present invention, the compounds were added to the cells in the presence of a fixed concentration (3.2×10⁻⁸ M) of the known RXR agonist LGD1069 (4-[(3,5,5,8,8-Pentamethyl-5,6,7,8-tetrahydro-2-naphthyl)ethenyl]benzoic acid: Ligand Pharmaceuticals, Inc.). Retinoid purity was established as greater than 99% by reverse phase high-performance liquid chromatography. Retinoids were dissolved in dimethylsulfoxide for use in the transcriptional activation assays. Two to three replicates were used for each sample. Transfections and subsequent procedures were performed on a Biomek 1000 automated workstation.

After 40 hours, the cells were washed with PBS, lysed with a detergent-based buffer and assayed for LUC and β-Gal activities using a luminometer or spectrophotometer, respectively. For each replicate, the normalized response (NR) was calculated as:

LUC response/β-Gal rate

where β-Gal rate=β-Gal·1×10⁵/β-al incubation time.

The mean and standard error of the mean (SEM) of the NR were calculated. Data were plotted as the response of the compound compared to the reference compounds over the range of the dose-response curve. For the agonist activity of the compounds of the present invention, the effective concentration that produced 50% of the maximum response (EC₅₀) was quantified. Antagonist activity was determined by testing the amount of LUC expression in the presence of the RXR agonists described above at the EC₅₀ concentration for such known compounds. The concentration of compounds of the present invention that inhibited 50% of LUC expression induced by the reference agonis was quantified (IC₅₀). In addition, the efficacy of antagonists was determined as a function (%) of maximal inhibition.

Table 2 below shows the activity of selected compounds of the present invention in terms of antagonist efficacy in the RXRα:RXRα homodimer cotransfection assay.

TABLE 2
Aantagonist efficacy in the RXRα:RXRα homodimer
cotransfection assay of select compounds of the invention.
RXR Antagonist CTF

| Example | IC50 (nM) | % Efficacy |
|---|---|---|
| 5 | 38 | 30 |
| 3 | 921 | 83 |
| 4 | | 29 |
| 1 | 4842 | 100 |
| 2 | 8712 | 100 |

### Example 6C

### RXR Heterodimer Co-Transfection Assays

The RXR modulator compounds of the present invention were further tested for activity on RXR heterodimers with RARα utilizing the cotransfection assay in CV-1 cells as described in Example 12B. The RXR:RAR heterodimer cotransfection assays utilized the following expression plasmids and reporter plasmid: pRShRARα (10 ng/well, Giguere et al., 330 Nature, 624 (1987) the disclosure of which is herein incorporated by reference) or pRShRARγ (10 ng/well, Ishikawa et al., 4 Mol. Endocrin, 837 (1990) the disclosure of which is herein incorporated by reference) with Δ-MTV-LUC (50 ng/well, Hollenberg and Evans, 55 Cell, 899 (1988), the disclosure of which is herein incorporated by reference) containing an RARE which is referred to as two copies of the TRE-palindromic response element described in Umesono et al., 336 Nature, 262 (1988), the disclosure of which is herein incorporated by reference. To conduct a RXR:PPARγ heterodimer cotransfection assay, the RXRα receptor expression plasmid, pRShRXRα (10 ng/well), can be cotransfected with the PPARγ expression plasmid, pCMVhPPARγ (10 ng/well), and a reporter plasmid containing three copies of a PPARγ response element (pPREA3-tk-LUC, 50 ng/well; Mukherjee et al. 272 Journ. Biol. Chem., 8071-8076 (1997) and references cited therein, the disclosures of which are herein incorporated by reference).

Cotransfections were performed as described in Example 12B. For determination of agonist activity in the context of the RXR:RAR heterodimer, media containing compounds of the present invention in concentrations ranging from 10⁻¹⁰ to 10⁻⁵ M were added to the cells. Similarly, the reference compounds all-trans retinoic acid (ATRA)(Sigma Chemical) and TTNPB ((E)-4-[2-(5,6,7,8-tetrahydro-5,5,8,8-tetramethyl-2-naphthalenyl)-1-propenyl]benzoic acid: Hoffman LaRoche, Inc.), known RAR agonist compounds were added at similar concentrations to provide a reference point for analysis of the agonist activity of the compounds of the present invention. Antagonist efficacy and IC₅₀ values were determined as in Example 12B.

RAR suppresses RXR ligand binding and transactivation of typical RXR agonists (e.g., LGD1069, LG100268) via allosteric interactions. Forman, B. M., Umesono, K., Chen, J., & Evans, R. M., Cell 81, 541-550 (1995) and Kurokawa, R., et. al. Nature 371, 528-531 (1994). However, when RAR is occupied, typical RXR agonists activate the heterodimer. Forman, B. M., Umesono, K, Chen, J., & Evans, R. M., Cell 81, 541-550 (1995) and Roy, B., Taneja, R., & Chambon, P., Mol. Cell. Biol. 15, 6481-6487 (1995). To examine the effects of the compounds of the present invention on the transcriptional properties of the RXR:RAR heterodimer, a heterodimer cotransfection assay as described above was employed. Table 3 below shows the activity of selected compounds of the present invention in terms of agonist efficacy in the RXR:RAR heterodimer cotransfection assay.

TABLE 3
Agonist efficacy in the RXRα:RXRα homodimer cotransfection
assay of select compounds of the invention.

RARa Synergy CTF

| Example | % Efficacy | fold induction |
|---|---|---|
| 5 | 7 | |
| 3 | 6 | 1 |
| 4 | 6 | 1 |
| 1 | 28 | 3 |
| 2 | 112 | 9 |

### Example 7

### Metabolic Study

A solution containing 1130 μL of 100 mM sodium phosphate buffer, pH 7.4, 20 μL of a 25 mg/mL CD-1 mouse liver microsomal suspension in 100 mM sodium phosphate buffer, pH 7.4, and 830 μL of a 4 mg/mL NADPH solution in 100 mM sodium phosphate buffer, pH 7.4, was prepared in a glass test tube, mixed on a vortexer, and incubated in a shaking water bath at 37° C. for 3 min. A test compound was dissolved in 10% DMSO/90% methanol to a final concentration of 400 μM, and 20 μL was added to the above solution after the 3 min. incubation. The solution was mixed on a vortexer, and incubated at 37° C. in the shaking water bath. After 0, 5, 10 and 20 min incubation, 75 μL aliquots of the incubation solution were removed in triplicate and each aliquot was added to a 75 μL solution that contained 2 μM of an internal standard in 50% acetonitrile/50% 20 mM ZnSO₄ and 20 mM NaOH in water. Samples were mixed on a vortexer, and centrifuged at 10° C. for 25 min at 3000 rpm. The supernatant was separated from the microsomal pellet and analyzed for the test compound by electrospray negative ionization using a Micromass Platform LCZ mass spectrometer equipped with a Shimadzu 10AD VP pump, and Shimadzu 10AD UP autosampler. Separation was achieved with a Phenomenex Luna phenyl-hexyl 3 micron (50×2mm) column and a methanol/5 mM ammonium acetate gradient. Peak area ratios of the test compound to internal standard at each time point were compared to the 0 min. time point to assess metabolic stability. A reference compound was treated in the same manner as the test compounds and the data was compared to determine whether the test compounds had improved metabolic stability.

TABLE 4
Metabolic stability of compounds of the invention.

| | Metabolic stability |
|---|---|
| | (mouse microsomes) |
| | Difference from reference |
| Example | compound at 20 min. (%) |
| 1 | 29.295 |
| 2 | 18.300 |

As shown in Table 4, the compounds of formula I in which at least one of R₈ or R₉ is F or at least one of R₅ or R₁₀ is fluoromethyl, difluoromethylor trifluoromethyl are substantially more stable than the reference compound.

### Example 8

### Evaluation of Activity In Vivo

Rodents that are genetically defective in the leptin pathway are commonly used as animal models of non-insulin dependant diabetes mellitus (NIDDM). db/db mice and ZDF rats develop frank diabetes that progresses to include β-cell failure and the accompanying precipitous drop in plasma insulin levels. Both strains are profoundly obese, hyperglycemic, hyperinsulinemic, and hypertriglyceridemic. fa/fa rats, on the other hand, are obese and insulin resistant but do not develop frank diabetes and the associated hyperglycemia. All three rodent models were used to examine the efficacy of oral dosing with compounds of the invention on diabetes, insulin sensitivity, food consumption and body weight gain.

Mice (obtained from Jackson Laboratory), ZDF rats (obtained from Genetic Models Inc.) and fa/fa rats (obtained from either Charles River, or Harlan) are maintained on 12-hour light/dark cycle. Mice (age 28-42 days) are caged in groups of 5-6. Rats (age 7 weeks) are housed individually. All animals are allowed ad libitum access to water and food (Purina 5015 for mice and 5008 for rats). Compounds are administered at the specified doses by oral gavage on the morning of each day of any experiment. Blood samples are obtained 3 hours after dosing from fed animals under anesthesia and collected into heparinized capillary tubes from the tail vein.

Mice transgenic for the human apolipoprotein A-I gene (obtained from Jackson Laboratory) are used to evaluate PPARγ mediated effects on high density lipoprotein (HDL) cholesterol. The mice are handled as described above for db/db mice, except that they are fed Purina 5001.

Compounds that are full agonists at the RXR homodimer, such as LG100268, are efficacious insulin sensitizers in rodent models of NIDDM and, thus, lower blood glucose levels. However, such compounds raise triglycerides and suppress the thyroid hormone axis in these animals. On the other hand, full antagonists have no effect on glucose, triglycerides or the thyroid status in these same model systems. We have identified a specific subset of rexinoids that maintain the desirable insulin sensitizing activity and eliminate both the suppression of the thyroid axis and triglyceride elevations. These compounds are heterodimer selective modulators of RXR activity. They bind to RXR with high affinity (generally Kᵢ<50 nM) and produce potent synergistic activation of the RXR:PPARγ heterodimer. This synergistic activation of PPARγ in vitro is presumably a major determinant of the antidiabetic efficacy of compounds in vivo. To eliminate the undesirable increases in triglycerides and suppression of T4, the modulators must not significantly activate RXR:RAR heterodimers and must have substantial RXR:RAR antagonist activity. Examples 3-5 in Table 3 clearly demonstrate that compounds of the invention do not activate RXR:RAR heterodimers.

When administered to obese, insulin resistant db/db mice (100 mg/kg by daily oral gavage for 14 days), compounds of the invention lower plasma glucose. However, unlike fall agonists (e.g., LG100268), they do not increase triglycerides.

Four week old db/db mice are essentially normoglycemic, they have not yet developed hyperglycemia. Treatment of such mice with a compound of the invention (30 mg/kg by daily oral gavage) prevents the development of hyperglycemia. This treatment is expected to successfully control plasma glucose levels for up to 11 weeks (when the mice are 15 weeks old).

Treatment of 7 week old db/db mice with metformin (300 mg/kg by daily oral gavage) lowers plasma glucose. However the maximum effect is seen following the first week of treatment. Over 3 subsequent weeks the efficacy of metformin decreases. At this point, treatment with metformin plus the addition of a compound of the invention (100 mg/kg by daily oral gavage) is expected to lowered plasma glucose to the level of age matched lean. Thus, the RXR modulator could be efficacious in cases of secondary failure of metformin.

To determine whether compounds of the invention produce insulin sensitization, compounds of the invention can be administered to insulin resistant fa/fa rats (100 mg/Kg by daily oral gavage for 14 days. In response to the oral glucose challenge, both insulin and glucose is expected to rise significantly less in animals treated with a compound of the invention than in untreated control animals. Animals treated with a compound of the invention are expected to consume the same amount of food and gain the same amount of weight as vehicle treated control animals. When fa/fa animals are treated with a thiazolinedione insulin sensitizer, they consume significantly more food and gain significantly more weight than control animals. In contrast, animals treated with a combination of the thiazolidinedione and a compound of the invention are expected to consume the same amount of food and gain the same amount of weight as the control animals. Compounds of the invention are expected to block the thiazolidinedione induced increases in both food consumption and body weight gain.

When administered to transgenic mice carrying the human apo A-I gene, compounds of the invention are expected to increase HDL cholesterol. However, unlike LG100268 which also raises triglycerides, compounds of the invention are not expected to raise triglycerides. Compounds of the invention that are not RXR:RAR heterodimer agonist and have greater than 50% RXR:RAR antagonists activity do not raise triglycerides in the transgenic mouse model, consistent with their heterodimer selectivity. This effect is consistent with activation of PPARα and, in fact, in vivo these compounds synergize with the weak PPARα agonist fenofibrate.

### Example 15

### Evaluation of Teratogenicity In Vivo

Teratogenicity is commonly evaluated by examination of fetuses obtained by cesarean section from pregnant mice dosed daily with test compound between gestation days 6-18. A blind study can be conducted using time-mated female Crl:CD-10® (ICR)BR mice to evaluate potential developmental toxicity (teratogenicity) following administration of a compound of the invention at either 30 or 200 mg/kg-day by daily oral gavage for the specified 12 days of gestation. Each test group consists of 7-8 pregnant females and produced approximately 100 live fetuses per test group. As a positive control, pregnant female mice are treated with the retinoid LG100268 at a dose of either 30 mg/kg-day or 100 mg/kg-day. Teratogenicity can be observed in fetuses from mice treated with the LG100268 at both dosage groups. In contrast, no teratogenic effects are expected to be observed in fetuses from mice treated with a compound of the invention. Compared to controls dosed with vehicle, no effects are expected to be observed on the number of Corpora lutea, implantation sites, live or dead fetuses, early or late resorptions, fetal weight or sex, gross external morphology or visceral morphology of the cranial region in fetuses from mice treated with a compound of the invention at either dose. The highest dose of a compound of the invention tested (200 mg/kg-day) is twice the dose required to produce maximum antidiabetic activity in db/db mice (100 mg/kg-day).

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. The compound represented by the following structural formula: 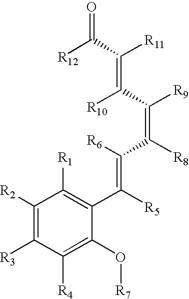
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

2. The compound of claim 1, wherein R₅ and R₆ are in a cis configuration.

3. The compound of claim 1, wherein R₇ is a C₂-C₅ alkyl which is optionally substituted with from one to nine fluoro groups.

4. The compound of claim 1, wherein R₂ and R₄ are the same and are isopropyl or t-butyl.

5. The compound of claim 1, wherein R₁₂ is OH.

6. The compound of claim 1, wherein:
R₅ and R₆ are in a cis configuration;
R₇ is a C₂-C₅ alkyl which is optionally substituted with from one to nine fluoro groups; and
R₁₂ is OH.

7. The compound of claim 1, wherein R₈ is F or R₁₀ is fluoromethyl, difluoromethyl, or trifluoromethyl.

8. The compound of claim 7, wherein R₅ and R₆ are in a cis configuration.

9. The compound of claim 7, wherein R₇ is a C₂-C₅ alkyl which is optionally substituted with from one to nine fluoro groups.

10. The compound of claim 7, wherein R₈ is H and R₁₀ is trifluoromethyl.

11. The compound of claim 7, wherein R₈ is F and R₁₀ is methyl.

12. The compound of claim 7, wherein R₁₂ is OH.

13. The compound of claim 7, wherein:
R₅ and R₆ are in a cis configuration;
R₇ is a C₂-C₅ alkyl which is optionally substituted with from one to nine fluoro groups; and
R₁₂ is OH.

14. A compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

15. A pharmaceutical composition, comprising a pharmaceutically acceptable carrier and at least one compound represented by the following structural formula: 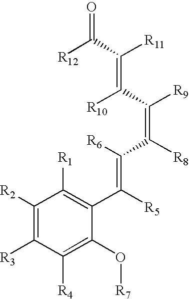
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkenyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

16. The pharmaceutical composition of claim 15, wherein R₅ and R₆ are in a cis configuration.

17. The pharmaceutical composition of claim 15, wherein R₇ is a C₂-C₅ alkyl which is optionally substituted with from one to nine fluoro groups.

18. The pharmaceutical composition of claim 15, wherein R₂ and R₄ are the same and are isopropyl or t-butyl.

19. The pharmaceutical composition of claim 15, wherein R₁₂ is OH.

20. The pharmaceutical composition of claim 15, wherein:
R₅ and R₆ are in a cis configuration;
R₇ is a C₂-C₅ alkyl which is optionally substituted with from one to nine fluoro groups; and
R₁₂ is OH.

21. The pharmaceutical composition of claim 15, wherein R₈ is F or R₁₀ is fluoromethyl, difluoromethyl, or trifluoromethyl.

22. The pharmaceutical composition of claim 21, wherein R₅ and R₈ are in a cis configuration.

23. The pharmaceutical composition of claim 21, wherein R₇ is a C₂-C₅ alkyl which is optionally substituted with from one to nine fluoro groups.

24. The pharmaceutical composition of claim 21, wherein R₈ is H and R₁₀ is trifluoromethyl.

25. The pharmaceutical composition of claim 21, wherein R₈ is F and R₁₀ is methyl.

26. The pharmaceutical composition of claim 21, wherein R₁₂ is OH.

27. The pharmaceutical composition of claim 21, wherein:
R₅ and R₆ are in a cis configuration;
R₇ is a C₂-C₅ alkyl which is optionally substituted with from one to nine fluoro groups; and
R₁₂ is OH.

28. A pharmaceutical composition compound, comprising a pharmaceutically acceptable carrier and at least one compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

29. A method for modulating retinoid X receptor activity in a mammal comprising administering to said mammal a pharmaceutically effective amount of at least one compound represented by the following structural formula: 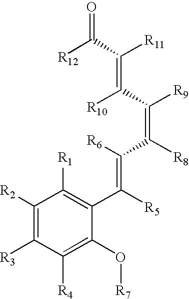
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

30. The method of claim 29, wherein R₅ and R₆ are in a cis configuration.

31. The method of claim 29, wherein R₇ is a C₂-C₅ alkyl which is optionally substituted with from one to nine fluoro groups.

32. The method of claim 29, wherein R₈ is F or R₁₀ is fluoromethyl, difluoromethyl, or trifluoromethyl.

33. The method of claim 29, wherein R₁₂ is OH.

34. The method of claim 29, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

35. A method for modulating RXRα:PPARα heterodimer activity in a mammal comprising administering to said mammal a pharmaceutically effective amount of at least one compound represented by the following structural formula: 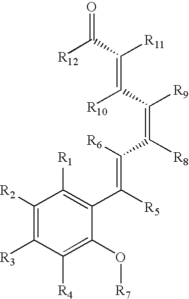
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈ , R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

36. The method of claim 35, wherein R₅ and R₆ are in a cis configuration.

37. The method of claim 35, wherein R₇ is a C₂-C₅ alkyl which is optionally substituted with from one to nine fluoro groups.

38. The method of claim 35, wherein R₈ is F or R₁₀ is fluoromethyl, difluoromethyl, or trifluoromethyl.

39. The method of claim 35, wherein R₁₂ is OH.

40. The method of claim 35, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

41. A method for modulating RXRα:PPARγ heterodimer activity in a mammal comprising administering to said mammal a pharmaceutically effective amount of at least one compound represented by the following structural formula: 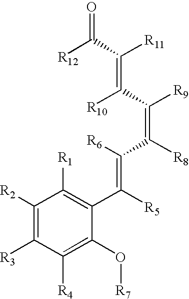
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

42. The method of claim 41, wherein R₅ and R₆ are in a cis configuration.

43. The method of claim 41, wherein R₇ is a C₂-C₅ alkyl which is optionally substituted with from one to nine fluoro groups.

44. The method of claim 41, wherein R₈ is F or R₁₀ is fluoromethyl, difluoromethyl, or trifluoromethyl.

45. The method of claim 41, wherein R₁₂ is OH.

46. The method of claim 41, wherein the compound selected from the group consisting of:
7-[3,5-di-tert-butyl-2-(2,2-difluoroethoxy)-phenyl]-4-fluoro3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

47. A method for increasing HDL cholesterol levels and reducing triglyceride levels in a mammal comprising administering to said mammal a pharmaceutically effective amount of at least one compound represented by the following structural formula: 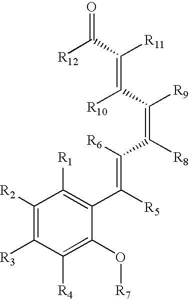
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

48. The method of claim 47, wherein R₅ and R₆ are in a cis configuration.

49. The method of claim 47, wherein R₇ is a C₂-C₅ alkyl which is optionally substituted with from one to nine fluoro groups.

50. The method of claim 47, wherein R₈ is F or R₁₀ is fluoromethyl, difluoromethyl, or trifluoromethyl.

51. The method of claim 47, wherein R₁₂ is OH.

52. The method of claim 47, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

53. A method for modulating lipid metabolizm in a mammal comprising administering to said mammal a pharmaceutically effective amount of at least one compound represented by the following structural formula: 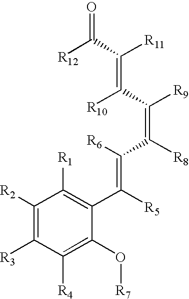
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

54. The method of claim 53, wherein R₅ and R₆ are in a cis configuration.

55. The method of claim 53, wherein R₇ is a C₂-C₅ alkyl which is optionally substituted with from one to nine fluoro groups.

56. The method of claim 53, wherein R₈ is F or R₁₀ is fluoromethyl, difluoromethyl, or trifluoromethyl.

57. The method of claim 53, wherein R₁₂ is OH.

58. The method of claim 53, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

59. A method for lowering blood glucose levels without altering serum triglyceride levels in a mammal comprising administering to said mammal a pharmaceutically effective amount of at least one compound represented by the following structural formula: 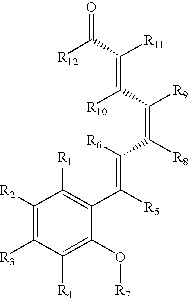
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy,
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

60. The method of claim 59, wherein R₅ and R₆ are in a cis configuration.

61. The method of claim 59, wherein R₇ is a C₂-C₅ alkyl which is optionally substituted with from one to nine fluoro groups.

62. The method of claim 59, wherein R₅ is F or R₁₀ is fluoromethyl, difluoromethyl, or trifluoromethyl.

63. The method of claim 59, wherein R₁₂ is OH.

64. The method of claim 59, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

65. A method treating or preventing a disease or condition selected from the group consisting of syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness in a mammal comprising administering to said mammal a pharmaceutically effective amount of a compound represented by the following structural formula: 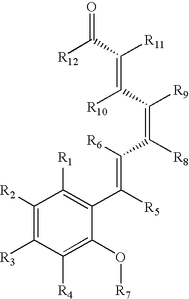
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C6 haloalkenyl, a heteroalkenyl, an optionally substituted C₂-C₆ alkynyl, C₂-C6 haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

66. The method of claim 65, wherein R₅ and R₆ are in a cis configuration.

67. The method of claim 65, wherein R₇ is a C₂-C₅ alkyl which is optionally substituted with from one to nine fluoro groups.

68. The method of claim 65, wherein R₈ is F or R₁₀ is fluoromethyl, difluoromethyl, or trifluoromethyl.

69. The method of claim 65, wherein R₁₂ is OH.

70. The method of claim 65, wherein the compound selected from the group consisting of:
7-[3,5-di-tert-butyl-2-(2,2-difluoroethoxy)-phenyl]-4-fluoro-3-methyl-octa-2,4,6-trienoic acid;
7-[3,5-di-tert-butyl-2-(2,2-difluoroethoxy)-phenyl]-5-fluoro-3-methyl-octa-2,4,6-trienoic acid;
(2Z,4E,6Z)-7-(2-butoxy-3,5-diisopropylphenyl)-3-trifluoromethyl-octa-2,4,6-trienoic acid;
(2E,4E,6Z)-7-(2-butoxy-3,5-diisopropylphenyl)-3-trifluoromethyl-octa-2,4,6-trienoic acid;
(2E,4E,6E)-3-methyl-7-(2-ethoxy-3,5-di-tert-butylphenyl)-8,8,8-trifluoroocta-2,4,6-trienoic acid;
and pharmaceutically acceptable salts, solvates and hydrates thereof.

71. A compound for use in therapy for a disorder modulated by a retinoid X receptor, a RXRα:PPARα heterodimer, or RXRα:PPARγ heterodimer, wherein the compound is represented by the following structural formula: 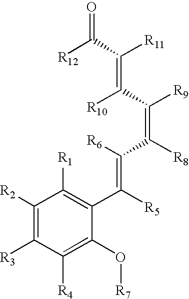
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

72. Use of a compound for the manufacture of a medicament for the treatment of a condition modulated by a retinoid X receptor, a RXRα:PPARα heterodimer, or RXRα:PPARγ heterodimer, wherein the compound is represented by the following structural formula: 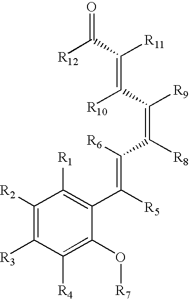
and geometrical isomers and pharmaceutically acceptable salts, solvates and hydrates thereof, wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R₁₂ is OR₁₅, OCH(R₁₇)OC(O)R₁₆, NR₁₇R₁₈ or an aminoalkoxy;
R₁₃ and R₁₄ are each, independently, H or an C₁-C₆ alkyl or taken together with the nitrogen to which they are attached form a heterocycle;
R₁₅ is H or a C₁-C₆ alkyl, an aryl or an aralkyl;
R₁₆ is a C₁-C₆ alkyl, an aryl or an aralkyl; and
R₁₇ and R₁₈ are each, independently, H, a C₁-C₆ alkyl, an aryl or an aralkyl.

73. A method of preparing a 7-(substituted phenyl)-3-methyl-octa-2,4,6-trienoic acid ester represented by the following structural formula: 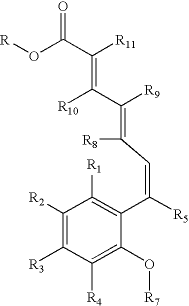
wherein:
R₁ is H or a halo;
R₂ and R₄ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy; or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₈, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R is a C₁-C₆ alkyl; and
R₁₃ and R₁₄ are each, independently, H or an C₁-C₆ alkyl or taken together with the nitrogen to which they are attached form a heterocycle, comprising the steps of:
a) reacting a substituted iodobenzene represented by the following formula: 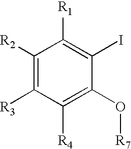
with a trimethyl silyl acetylene to form a (substituted phenyl)-trimethylsilane represented by the following structural formula: 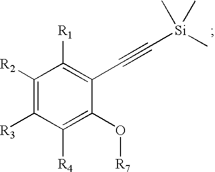
b) reacting the (substituted phenyl)-trimethylsilane with nickel(II)acetylacetonate and a dimethyl zinc represented by the formula Zn(R₅)₂ to form a [(substituted phenyl)-propenyl]-trimethylsilane represented by the following structural formula: 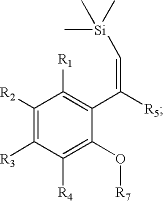
c) reacting the [(substituted phenyl)-propenyl]-trimethylsilane with iodine monochloride to form a (2-iodo-1-methylvinyl) benzene represented by the following structural formula: 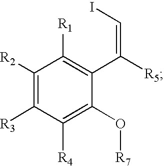
d) reacting a methyl phenyl sulfone represented by the following structural formula: 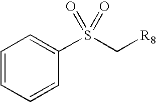
with a dialkylchlorophosphate represented by the following structural formula: 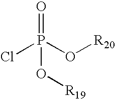
to form a sulfone reagent represented by the following structural formula: 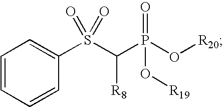
e) reacting a 3-methyl4-oxocrotonate represented by the following structural formula: 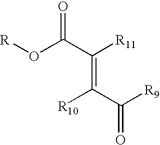
with the sulfone reagent to form a 5-benzensulfonyl-methyl represented by the following structural formula: 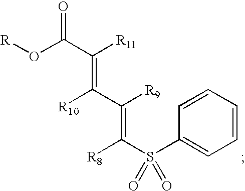
f) reacting the 5-benzensulfonyl-methyl with tributyl tin hydride and a free radical initiator to form a 3-methyl-5-tributylstannayl-penta-2,4-dienoic acid alkyl ester represented by the following structural formula: 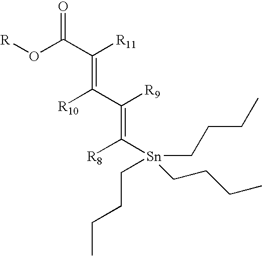
g) reacting the (2-iodo-1-methyl-vinyl) benzene and the 3-methyl-5-tributylstannayl-penta-2,4-dienoic acid alkyl ester in the presence of a catalytic amount of dichlorobis(triphenylphosphine)palladium(II) to form said 7-(substituted phenyl)-3-methyl-octa-2,4,6-trienoic acid ester.

74. The method of claim 73, further comprising the step of treating the 7-(substituted phenyl)-3-methyl-octa-2,4,6-trienoic acid ester with an alkali metal hydroxide to form a 7-(substituted phenyl)-3-methyl-octa-2,4,6-trienoic acid.

75. A method of preparing a 7-(substituted phenyl)-3-methyl-octa-2,4,6-trienoic acid ester represented by the following structural formula: 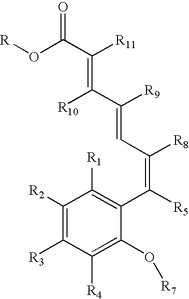
wherein:
R₁ is H or a halo;
R₂ and R₄ are each, independently, H, an optionally substituted C₁-C6 alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₃R₁₄; and
R₃ is H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or
R₂ and R₃ or R₃ and R₄ taken together with the carbons to which they are attached form an optionally substituted five, six or seven membered carbocyclic or heterocyclic ring; and
R₅ and R₁₀ are each, independently, methyl, fluoromethyl, difluoromethyl, or trifluoromethyl;
R₆, R₉ and R₁₁ are each, independently, H or F;
provided that at least one of R₈ or R₉ is F, or at least one of R₅ or R₁₀ is fluoromethyl, difluoromethyl, or trifluoromethyl;
R₇ is an optionally substituted C₁-C₆ alkyl, an optionally substituted C₂-C₅ alkenyl, a C₁-C₆ haloalkyl, an optionally substituted aryl or an optionally substituted heteroaryl;
R is a C₁-C₆ alkyl group;
R₁₃ and R₁₄ are each, independently, H or an C₁-C₆ alkyl or taken together with the nitrogen to which they are attached form a heterocycle, wherein R₅ and R₆ are in a cis configuration, comprising the steps of:
a) treating a trialkyl phosphonoacetate represented by the following structural formula: 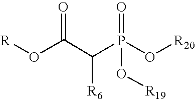
wherein R₁₉ and R₂₀ are each, independently, a C₁-C₆ alkyl, with sodium hydride to form an anion;
b) reacting the anion of the trialkyl phosphonoacetate with a 2-acetylphenol represented by the following structural formula: 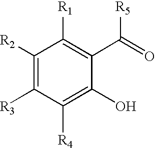
to form a coumarin represented by the following structural formula: 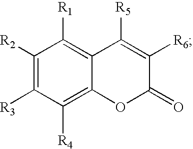
c) reacting the coumarin with a reducing agent to form a 2-(4-hydroxybut-2-en-2-yl) phenol represented by the following structural formula: 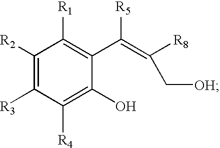
d) reacting the 2-(4-hydroxybut-2-en-2-yl) phenol with an aliphatic halide represented by the formula R₇-X in the presence of cesium fluoride or cesium carbonate to form a 3-(substituted phenyl)-but-2-en-1-ol represented by the following structural formula: 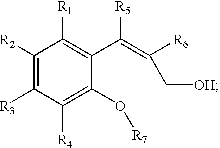
e) oxidizing the 3-(substituted phenyl)-but-2-en-1-ol with 4-methylmorpholine N-oxide in the presence of tetrapropylammonium perruthenate to form a 3-(substituted phenyl)-but-2-en-1-al represented by the following structural formula: 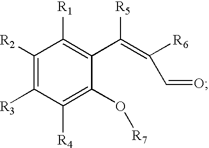
f) reacting a trialkyl 3-methylphosphocrotonate represented by the following structural formula: 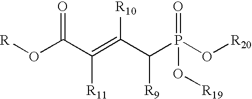
with an alkyl lithium to form an anion;
g) reacting the anion of the trialkyl 3-methylphosphocrotonate with the 3-(substituted phenyl)-but-2-en-1-al to form said 7-(substituted phenyl)-3-methyl-octa-2,4,6-trienoic acid ester.

76. The method of claim 75, further comprising the step of treating the 7-(substituted phenyl)-3-methyl-octa-2,4,6-trienoic acid ester with an alkali metal hydroxide to form a 7-substituted phenyl)-3-methyl-octa-2,4,6-trienoic acid.
